(19) United States
(12) Patent Application Publication
(10) Pub. No.: US 2010/0036319 A1
(43) Pub. Date: Feb. 11, 2010

(54) AUTO INJECTOR

(75) Inventors: David Drake, Bassingbourn Herts (GB); Douglas Ivan Jennings, Royston Herts (GB); Stuart David Cooper, Cambridgeshire (GB); Timothy Donald Barrow-Williams, Oxon (GB); Joseph Peter Corrigan, Cambridge (GB); Rosalind Trudy Lee, Cambridge (GB); David Whitaker, London (GB)

Correspondence Address: FOLEY AND LARDNER LLP;SUITE 500, 3000 K STREET NW, WASHINGTON, DC, 20007, US

(73) Assignee: PA KNOWLEDGE LIMITED, London (GB)

(21) Appl. No.: 12/525,348

(22) PCT Filed: Jan. 28, 2008
(86) PCT No.: PCT/GB08/00287

§ 371 Date: Sep. 2, 2009

(30) Foreign Application Priority Data
Feb. 1, 2007 (GB) 0701964.9

(51) Int. Cl. A61M 5/20; B23P 11/00

(52) U.S. Cl. 604/135; 29/428

(57) ABSTRACT

An auto injector including: a housing; an outlet portion with a needle (110) moveable relative to the housing; a container (104) within the housing for containing medicament; and an actuatable deployment mechanism configured to deploy the outlet portion by moving the outlet portion relative to the housing and to eject medicament contained in the container through the outlet portion. The auto injector is configured to store a volume of fluid and includes a filling mechanism configured to expel the volume of fluid from the outlet portion and subsequently to draw medicament through the outlet portion and into the container. An adaptor (112) fits to the auto injector so as to cover the needle and includes a puncture member configured to puncture a vial, the puncture member providing fluid communication to the needle. The auto injector and adaptor are provided as part of an auto injector pack together with a vial containing a substance which in combination with the liquid forms the medicament and a casing configured to house the vial, the auto injector and the adaptor

The present invention relates to an auto injector, in particular to an auto injector where a medicament is to be drawn into the auto injector and more particularly where a fluid, such as water, is to be mixed with another substance to form the medicament. It also relates to an adaptor, an auto injector set, an auto injector pack and a method of preparing an auto injector.

Various arrangements have been proposed for automatically deploying the needle of a syringe and then dispensing its contents, for instance as described in WO 95/35126, EP-A-0 517 473, U.S. Pat. No. 6,159,181 and WO 03/092771.

It is also known to provide medicaments in the form of a freeze-dried or lyophilised “cake”. The lyophilised medicament may be held within an impervious container, such as a glass vial or syringe. For injection purposes the medicament is reconstituted with water, usually sterile water for injection (sWFI).

The reconstitution step may be carried out manually, for instance, by injecting water into a sealed vial containing the medicament and then withdrawing the resulting solution back into the syringe. The medicament solution may then be injected directly from this syringe or transferred to an alternative injection device.

Noting that such arrangements are awkward and involved for the operators, WO 2006/131756 proposes a development to the earlier auto injectors mentioned above. In particular, the dispensing cylinder within the auto injector is divided into two compartments. Lyophilised medicament is provided in the front compartment and water is provided in the rear compartment. A moveable seal is provided between the two compartments, the movable seal including a one-way valve. When the auto injector is actuated such that the cylinder is driven forwards, a seal containing the one-way valve is moved backwards relative to the cylinder such that the water in the rear compartment moves to the front compartment so as to mix with the lyophilised medicament. Subsequent movement of a main piston in the cylinder drives the medicament solution out of the cylinder for injection purposes.

Although such auto injectors are effective in use, they have a number of limitations, for instance complexity of construction. There are also difficulties and costs in manufacture resulting from the need to provide different lines of production for different medicaments and difficulties in ensuring correct separation of the lyophilised medicament and water within the device.

It is an object of the present invention at least to reduce drawbacks with earlier systems and provide an alternative arrangement providing its own advantages.

The present invention is based partly on at least a recognition of the possibility of providing an auto injector which can be filled with the required medicament by the user and, more particularly, to an auto injector which comes pre-filled with fluid, such as water, for use in forming that medicament.

According to the present invention, there is provided an auto injector including:
a housing;
an outlet portion moveable relative to the housing;
a container within the housing for containing medicament;
an actuatable deployment mechanism configured to deploy the outlet portion by moving the outlet portion relative to the housing and to eject medicament contained in the container through the outlet portion; wherein:
the auto injector includes:
a filling mechanism configured to draw medicament through the outlet portion and into the container.

Preferably, the outlet portion is configured to hold a needle such that the needle is in fluid communication with a passageway included in the outlet portion.

It is possible for a needle to be fitted to the outlet portion only prior to actuation of the deployment mechanism for ejecting medicament contained in the container. In this case, the outlet portion can be used without a needle to expel the volume of fluid and draw medicament back into the container. A separate passageway could be provided for this purpose and/or a separate needle.

Preferably, however, the outlet portion includes the needle in fluid communication with the passageway. This needle could be used for expelling the volume of fluid and drawing in medicament as well as for ejecting medicament or could be replaced by a new needle for ejecting the medicament.

The container preferably takes the form of a standard syringe, for instance including walls defining an elongate bore extending from a container outlet and a piston sealing with the walls and slidable along the bore.

In such an arrangement, the deployment mechanism is configured to eject medicament contained in the container by sliding the piston along the bore towards the container outlet.

The deployment mechanism may be configured in any known manner according to earlier auto injector arrangements.

Preferably, the filling mechanism is configured to draw medicament into the container by sliding the piston along the bore away from the container outlet.

Preferably, the filling mechanism includes a filling member movable by a user from a first position to a second position to draw medicament through the outlet portion and into the container.

Preferably, the filling member is configured as a sleeve around the housing and is configured to slide along the housing from the first position to the second position.

This provides a convenient and effective way of operating the filling mechanism.

Preferably, the deployment mechanism includes a user actuatable deployment actuator configured to actuate the deployment mechanism.

The auto injector can be provided with a single actuator operating both as the filling actuator and the deployment actuator.

The filling mechanism can include a lock-out component for preventing operation of the user actuatable deployment actuator until the filling mechanism has drawn medicament into the container.

This ensures that a user does not operate the deployment mechanism if the filling operation has been completed.

The filling member can be configured to cover the user actuatable deployment member in the first position and to expose the user actuatable deployment member in the second position.

In this way, the filling member provides the lock-out and prevents actuation of the deployment mechanism until after medicament has been drawn into the container.

Preferably, the auto injector is configured to store a volume of fluid and the filling mechanism is configured to expel the volume of fluid from the outlet portion before drawing medicament through the outlet portion and into the container.

Thus, the auto injector can come pre-filled with fluid for use in forming a medicament. The previous auto injectors are configured only to expel their contents as part of the injection step and then, in some arrangements, withdraw the needle of the auto injector back into the housing. However, according to the present invention, the auto injector is capable of ejecting the fluid, for instance into a vial of lyophilised medicament, and draw back into the container the required medicament, for instance formed by the fluid mixing with the lyophilised medicament in the vial. In this way, a single auto injector can be provided for many different uses with many different types of medicament. It is possible to construct an auto injector more simply without the two compartments of the prior art and to manufacture the auto injector without concerns of fluid and medicament within the auto injector coming into contact with one another.

It would be possible for the volume of fluid within the auto injector to be stored in its own compartment prior to being expelled by the filling mechanism. However, preferably, the container is configured to store the volume of fluid.

Conceivably, the auto injector could be used with any type of fluid or liquid, but preferably the fluid is water or a buffer and the auto injector may be provided including water already stored in the container.

Where the container is also used to store the volume of fluid, the filling mechanism is configured to expel the volume of fluid also by sliding the piston along the bore towards the container outlet.

As will be considered below, this operation could be conducted according to power provided within the auto injector or could be conducted manually by a linkage allowing the user to slide the piston along the bore.

An internal power source may be provided to power the filling mechanism to expel the volume of fluid. Similarly, any internal power source could be provided to power the filling mechanism to draw medicament into the container. However, preferably, a power source for powering the filling mechanism to expel the volume of fluid is additionally configured to power the deployment mechanism. With this arrangement, it is particularly advantageous if, having used the power of the power source to expel the volume of fluid, manual operation by the user to draw medicament into the container additionally replenishes power to the power source which can then be subsequently used to power the deployment mechanism.

In this respect, the power source is preferably a spring, The energy of the spring may be released when expelling the volume of fluid, restored manually by the user when drawing medicament into the container and then released again when powering the deployment mechanism.

Preferably, the filling mechanism includes a user actuatable filling actuator configured to actuate the filling mechanism to expel volume of fluid.

The filling member may be a member for manually moving components to operate the filling mechanism or may be a member merely for releasing the internal power source.

The present invention in its broadest sense relates to providing an auto injector which is able to draw medicament into its container prior to its deployment. It may be pre-filled with a fluid for use in forming a medicament, that fluid being ejected to mix with a substance to form the medicament and then drawn back into the auto injector for subsequent use.

Unfortunately, there can be a problem in using the needle of the auto injector to puncture the septum of a vial containing either the ready medicament or the substance for the medicament to be mixed with the liquid in the auto injector. In particular, puncturing the septum of the vial with the needle will blunt the tip of the needle. Also, there is a danger that a small portion of the septum may become detached during the puncturing process and will remain on the needle as a subsequent danger to any patient using the needle.

It is possible for a user to use a first needle to puncture the septum of a vial and obtain the mixed medicament and then exchange the first needle for a second needle before the auto injector is used for dispensing the medicament to a patient. As an alternative, something described as a “straw” could be used instead of the first needle to puncture the septum of the vial.

Exchanging the straw or needle for the injection needle will be troublesome to the user and increases the risk of accidental injury from the needle. Also, with arrangements of auto injectors where the needle is generally kept hidden, gaining access to the needle to exchange it will be difficult.

Furthermore, operation of using a needle to pierce a vial septum will require a level of skill that not all patients will necessary have or may not be comfortable with when the medicament is required. As a result (due to varying depths in which a septum is pierced by a needle) it is possible that a user can influence the process of injecting the fluid into, or withdrawing fluid from, a vial and so affect the constitution or quantity of the medicament delivered.

In view of the above, the present invention also proposes an adaptor for use with an auto injector having a container having an elongate body with an injection end from which a needle extends to a needle tip, the adaptor including:
a housing arranged to mount on and seal with the container so as to define with the container a cavity within which the needle of the container can extend without the needle tip contacting the housing; and
a puncture member extending from the housing, arranged to puncture a septum of a vial sealing an interior of the vial and having at least one port arranged to provide fluid communication between the interior of the vial and the cavity.

In this way, the needle of the auto injector can be retained in a state useful for penetrating the skin of a patient for injection purposes. In particular, the tip of the needle is prevented from contacting any surfaces which might blunt it. The puncture member of the adapter is used to puncture the septum of a vial so that any stray material from the septum remains on the puncture member rather than the needle and the needle is not made blunt by the puncturing process. The adaptor provides fluid communication between the interior of the vial and the auto injector such that the contents of the vial can be withdrawn into the auto injector. The user then need only remove the adaptor from the auto injector to place the auto injector in a state ready for use. There is no need to exchange the needle fitted to the auto injector with the other needle.

The housing of the adaptor preferably includes a mounting portion for fitment to the housing of the auto injector so as to secure the adaptor to the auto injector.

In this way, it is not necessary for the seal with the container within the auto injector to provide any physical support to the adaptor. The adaptor may be held securely in place by means of the mounting portion during its use.

The mounting portion may include a collar for fitment to the housing of the auto injector. The housing of the adaptor then extends within the collar to mount on and seal with the container of the auto injector.

The collar preferably mounts to an outer surface of the housing of the auto injector, but could also or alternatively mount to an inner surface.

Preferably, the mounting portion, once fitted to an auto injector, prevents axial movement of the adaptor relative to the auto injector. In this regard, the mounting portion may include one of a bayonet fitting and a threaded fitting.

The present invention may also provide an auto injector set including an auto injector having a needle with which to inject medicament; and an adaptor configured to fit to the auto injector so as to cover the needle, the adaptor including a puncture member configured to puncture a vial, the puncture member providing fluid communication to the needle.

Preferably, the adaptor is constructed in the manner described above.

The housing of the auto injector preferably includes a receiving portion on which to fit the adaptor. In particular, where the adaptor includes a mounting portion, the receiving portion receives the mounting portion.

Preferably, the receiving portion interacts with the adaptor so as to prevent axial movement between the adaptor and the auto injector. In this respect, the receiving portion may include one of a bayonet fitting and a threaded fitting.

Preferably, the auto injector can be constructed in the manner described above.

Preferably the adaptor has a housing arranged to mount on and seal with the auto injector so as to define with the auto injector a cavity within which the needle of the auto injector can extend without the needle tip contacting the adaptor housing and a puncture member extending from the adaptor housing, arranged to puncture a septum of a vial sealing an interior of the vial and having at least one port arranged to provide fluid communication between the interior of the vial and the cavity.

The container within the auto injector can take the form of a syringe body. It is possible for the needle to be provided in the injection end of the syringe body in a fixed manner.

It is possible for the housing of the adaptor to seal with the shaft of the needle of the syringe body so as to form the cavity which connects to the at least one port. However, with such an arrangement, steps should be taken to avoid any seal from contacting the tip of the needle.

Preferably, however, the housing of the adaptor has a substantially circular opening for sealing with the injection end of the syringe body. In this way, it is not necessary for the housing of the adaptor to have any contact with the needle.

Preferably, where the adaptor is intended for use with an auto injector having, at the injection end of the syringe body, a neck portion from which the needle extends, the housing of the adaptor is arranged to seal with the neck portion.

As will be discussed below, a limited number of highly standardised syringe bodies are well known and it is easy to arrange the adaptor for fitment and sealing with such syringe bodies. The auto injector can incorporate such a syringe body pre-filled with the fluid.

Preferably, the housing of the adaptor includes an inwardly facing O-ring for sealing with the syringe body. This could seal with the shaft of the needle, but, preferably, seals with the neck portion. Sealing with the shape of the needle can be advantageous in reducing lost volume.

Preferably, the puncture member extends from the housing of the adaptor to a puncture portion shaped to puncture a vial septum, the at least one port being located at least proximate to the puncture portion and wherein the puncture member includes an internal surface defining a communication channel internally of the puncture member and extending from the at least one port to the cavity.

Thus, the puncture portion at the end of the puncture member can be used to puncture a vial septum. With the puncture portion then inside the vial and the at least one port located proximate to it, fluid communication is provided between the inside of the vial and the puncture member. The communication channel provided within the puncture member then connects the at least one port to the cavity so as to provide full fluid communication between the inside of the vial and the cavity of the adaptor.

Preferably, the puncture member includes an outer surface extending longitudinally from the adapter housing to the puncture portion, the at least one port being defined in the outer surface at a position proximate to the puncture portion.

In other words, the puncture member preferably has an elongate form such as in the shape of a shaft, with the puncture portion at one end. The at least one port is then defined or provided through the outer surface of that elongate form at a position proximate to the puncture portion.

Preferably, at least part of the communication channel forms part of the cavity such that the needle can extend into the communication channel without the needle tip contacting the adapter housing or puncture member.

Thus, the division between the communication channel and the cavity is somewhat blurred. Both internal spaces are provided within the adaptor and are connected to one another. When the adaptor is connected to a syringe body, the needle extends through the space considered as the cavity and into the space considered as the communication channel where both of these spaces are in fluid communication with the at least one port.

Preferably, the communication channel is substantially cylindrical and is aligned with the adaptor housing so as to receive the needle along substantially the length of the puncture member.

Thus, the space considered to be the cavity may be kept very small with the needle extending internally along the length of the puncture member.

Preferably, the communication channel, cavity and adapter housing are all coaxial. It will be appreciated that some standard syringes have needles arranged coaxially with the elongate body of the syringe such that this arrangement of the adaptor allows the adaptor to be fitted coaxially with the syringe.

Preferably, the internal surface of the puncture member is arranged to be close fitting to the needle and the adaptor housing is arranged to be close fitting to the injection end such that the volume or space within the adaptor, between the adaptor and the syringe body, is small.

Preferably, the volume or space should be made as small as possible according to tolerances of manufacture whilst ensuring that the tip of the needle does not contact either the puncture member or the adaptor housing. The space or volume within the adaptor will be dead volume where water or medicament is lost. Preferably, this volume should be arranged to be no more than 5% of the volume of medicament for injection or volume of water for injection into the vial. Hence, for a total volume of 1 millilitre, the lost volume should be no more than 50 micro litres. 3% or 1% are more preferable values.

Preferably, the at least one port includes two or more ports. These may be provided at different radial positions around the puncture member and/or different longitudinal positions along the length of the puncture member.

Preferably, the housing of the adaptor includes an attachment member extending substantially parallel with the puncture member so as to engage with a neck of a vial.

It will be appreciated that a range of standard vials are well known having a neck leading to an opening covered by the vial septum.

By allowing the adaptor to engage with the neck of a vial, the vial can be supported more securely to the adaptor so as to facilitate operation of the auto injector while the puncture member remains inserted through the septum of the vial.

Preferably, the attachment member extends on opposite sides of the puncture member.

This provides a convenient way of attaching to and gripping the neck of the vial. The attachment member may be substantially annular so as to grip the neck of the vial substantially around its entire periphery.

The puncture member should not contact the body of the vial, for instance the glass housing. Thus, preferably, the puncture member is arranged to pierce the septum close to the centre of the septum. The attachment member maybe arranged so as to ensure this positioning.

Preferably, the attachment member includes a latch for non-releasably engaging with the neck of the vial.

In this way, the adaptor may be fitted to a vial, for instance by push fitting the attachment member over the neck of the vial. Preferably during this operation, the puncture member punctures the septum. By non-releasably engaging with the neck of the vial, it then becomes easy for the user to place the auto injector in a state ready for use. In particular, the user merely pulls the auto injector with respect to the vial. The adaptor remains engaged with the neck of the vial and the auto injector pulls away from the adaptor.

The adapter is preferably arranged for use with one of the following standard types of vial:
Schott
Becton Dickinson
Qorpak
West Pharmaceutical (Westar closure components)
Tyco
Pacific Vial
These may be any of the standard vial sizes 0.3 ml, 0.5 ml, 1 ml, 1.5 ml, 2 ml, 3 ml, 4 ml, 5 ml, 10 ml, 20 ml, 40 ml, 60 ml.

The features and advantages of an auto injector pre-filled with fluid as discussed above, the present invention also recognises for the first time the possibility of providing an advantageous auto injector pack.

According to the present invention there is provided an auto injector pack including an auto injector set as described above, a vial containing a substance which in combination with the liquid forms the medicament and a casing configured to house the vial and also the auto injector and the adaptor of the auto injector set.

This provides a convenient combination of components which are ready for use.

Preferably, the casing includes a pre-sealed and sterilised section containing the vial and adaptor.

The vial and adaptor may be provided within a section which is, for instance sealed by foil and then subjected to gamma radiation during manufacture.

Preferably, the casing includes a lower portion having a shape to hold each of the auto injector, the vial and the adaptor individually, the lower portion having a shape to hold the adaptor facing outwardly so as to receive the needle of the auto injector and to hold the vial facing outwardly so as to receive the puncture member.

In this way, it is not necessary to remove the adaptor from the casing in order to fit the auto injector to it. A user may merely fit the auto injector to the adaptor with the adaptor held in place in the lower portion of the casing. Moving the auto injector away from the lower portion of the case will take with it the adaptor. The assembly of the auto injector and adaptor can then similarly be fitted to the vial with the vial still held in the lower portion of the casing.

This provides a very convenient arrangement for the user without the user having to hold and manipulate the adaptor and vial.

Preferably, the lower portion has a shape to hold the vial and adaptor together as an assembled unit after being detached from the auto injector.

The assembled unit of the vial and adaptor will of course be larger than either the vial or the adaptor individually and, hence, may not fit back into the casing in either the original position of the vial or the adaptor. By providing the casing with a shape to hold the assembled unit, the user is able to replace the vial and adaptor in the casing, for instance for overall disposal.

Preferably, the lower portion has the form of a tray, the auto injector extends along an axis from the needle to an actuator end and the lower portion has a shape to hold the auto injector with the axis of the auto injector in the tray.

This is a convenient way of storing an auto injector which has a substantial length in comparison to the length of the adaptor and vial.

Preferably, the casing includes a presentation mechanism configured to lift the actuation end of the auto injector out of the lower portion upon opening of the casing.

This is advantageous in providing a clear indication to the user of the orientation in which to lift the auto injector from the casing.

Preferably, the lower portion is shaped to hold the actuation end of the auto injector with the auto injector orientated substantially perpendicular to the tray.

In this way, having first fitted the adaptor to the auto injector and then fitted the vial to the adaptor, a user can turn the auto injector upside down and leave the auto injector in an upright orientation supported by its actuation end in the lower portion of the casing.

Preferably, fluid in the auto injector is ejected into the vial with the vial still held in the lower portion of the casing. Removing the total assembly and turning it upside down helps mix the fluid with the substance in the vial and locating the actuation end downwards in the lower portion provides a good orientation for drawing the medicament from vial into the auto injector.

Preferably, the casing includes an upper portion forming a cover openable with respect to the lower portion.

The inner surface of the upper portion can be provided with instructions for use of the auto injector, adaptor and vial.

The present invention also provides a method of preparing the auto injector of the auto injector pack, the method including: removing the auto injector from the casing; fitting the auto injector to the adaptor while the adaptor is in the casing; removing the adaptor from the casing while fitted to the auto injector; while the vial is in the casing, puncturing the vial with the puncture member of the adaptor and ejecting the fluid from the auto injector through the adaptor into the vial; removing the vial from the casing while fitted to the adaptor and auto injector; reorientating the auto injector and fitting the auto injector to the casing with the vial and adaptor above the auto injector; withdrawing the medicament formed in the vial through the adaptor and into the auto injector; and removing the adaptor and vial from the auto injector.

The invention will be more clearly understood from the following description, given by way of example only, with reference to the accompanying drawings, in which:

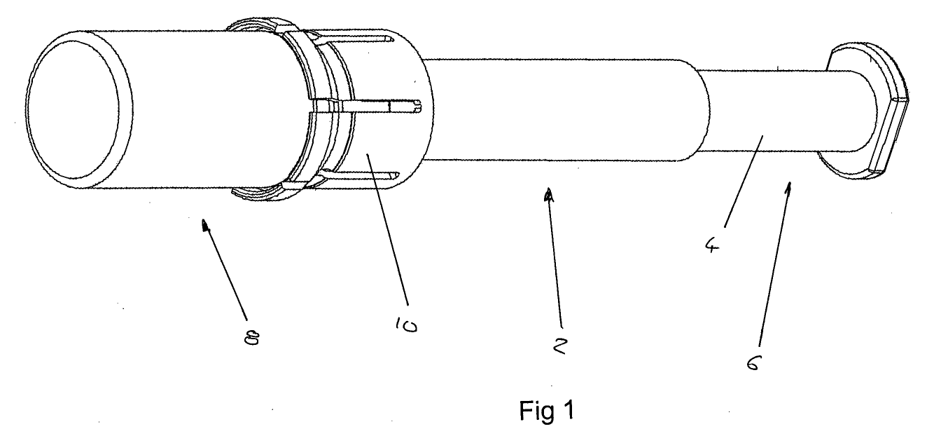

FIG. 1 illustrates an adaptor fitted to a syringe and a vial;

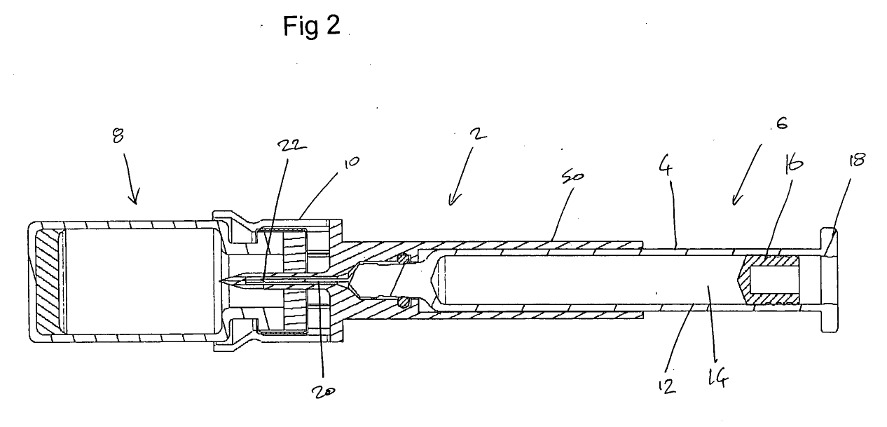

FIG. 2 illustrates a cross-section through the assembly of FIG. 1;

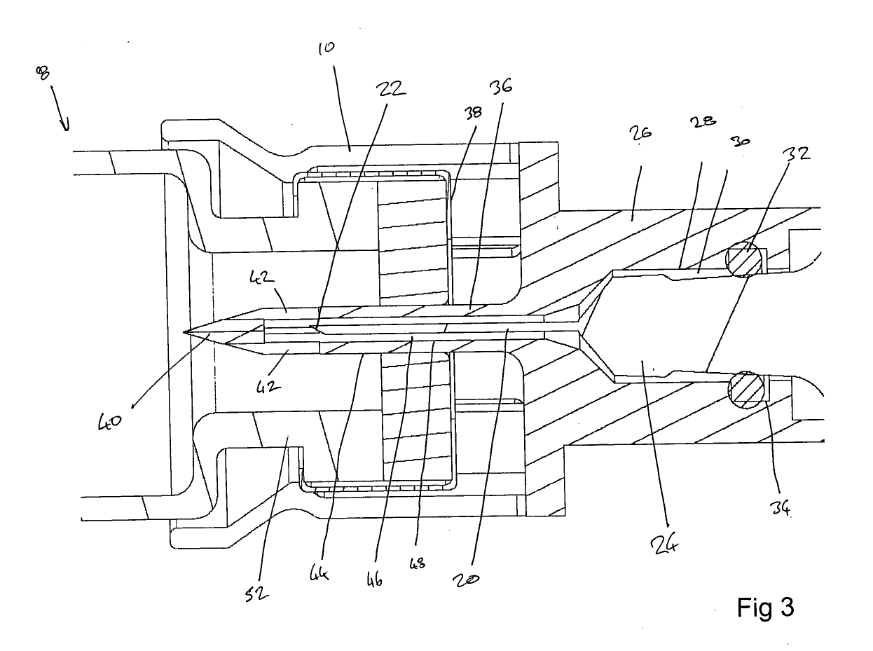

FIG. 3 illustrates an enlarged section of FIG. 2;

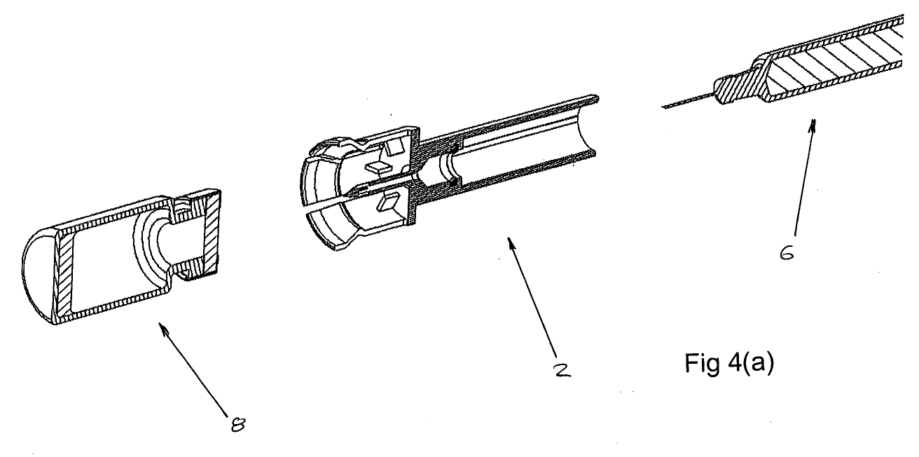

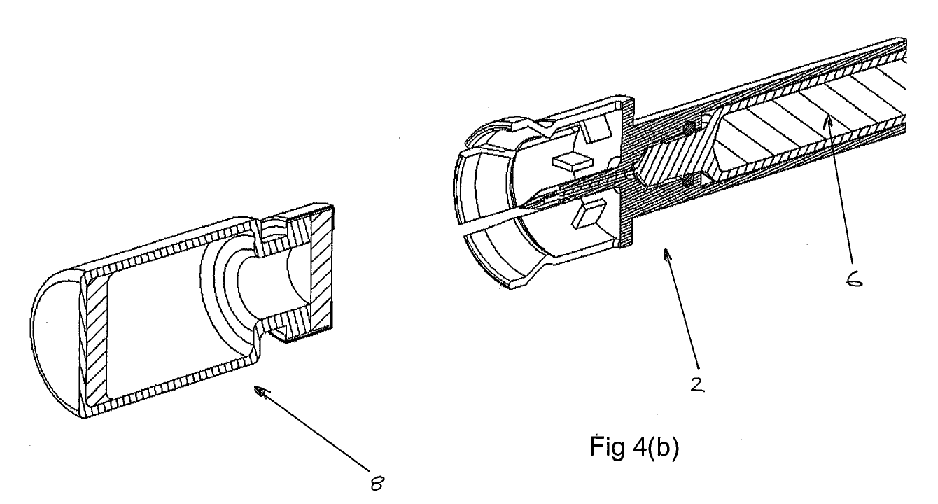

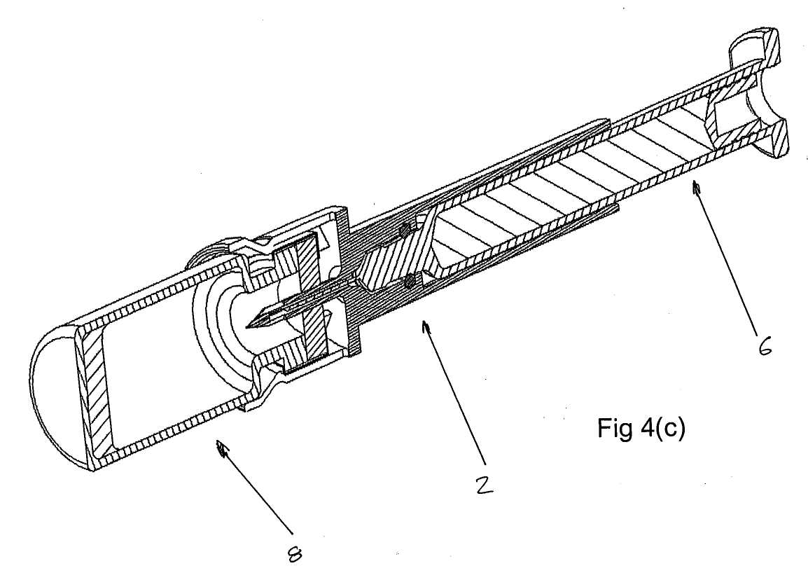

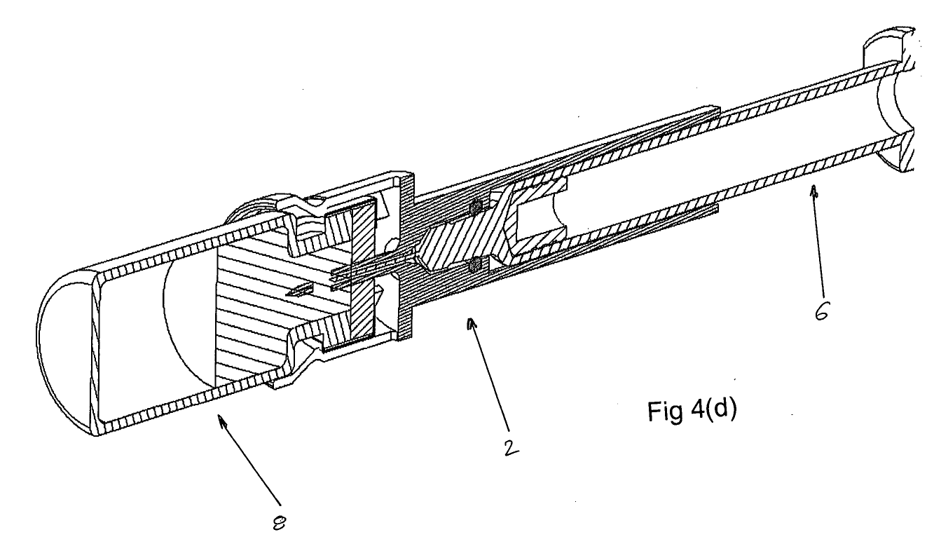

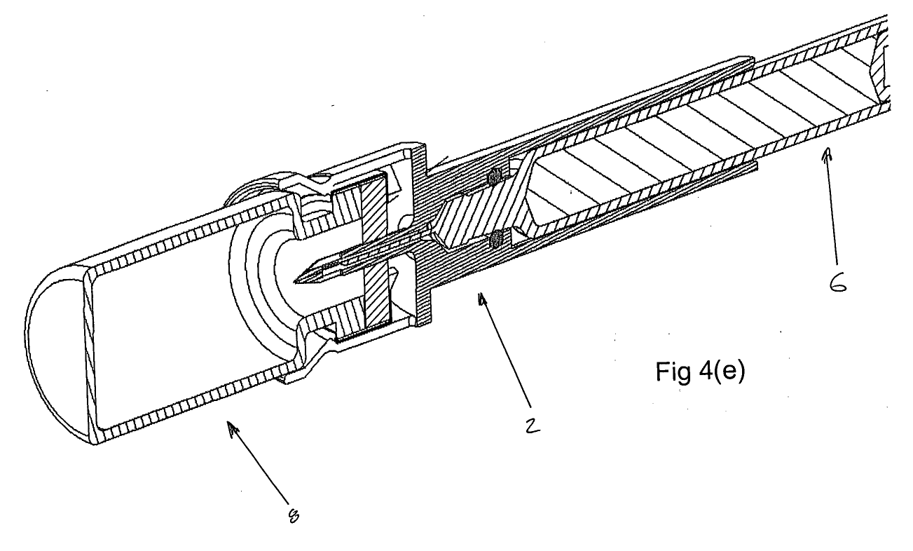

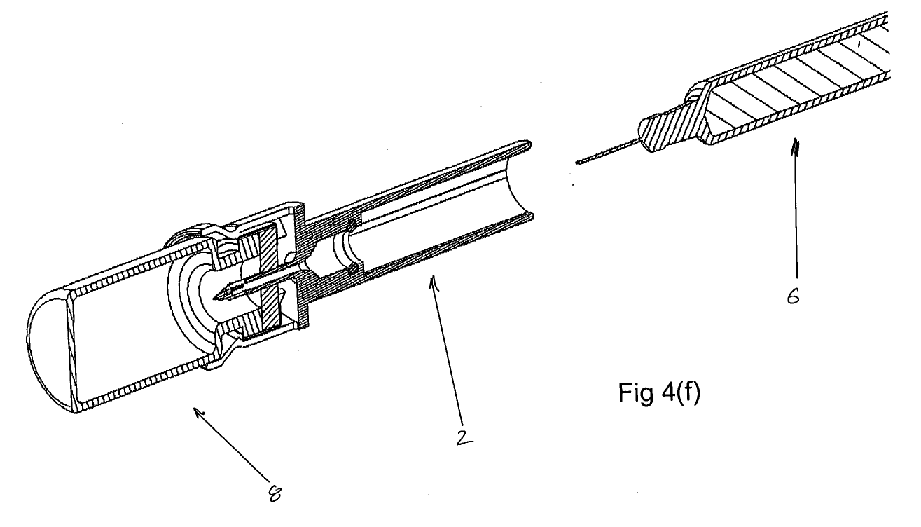

FIGS. 4(a) to (i) and 5(a) to (f) illustrate various stages of operation of the arrangement of FIG. 1;

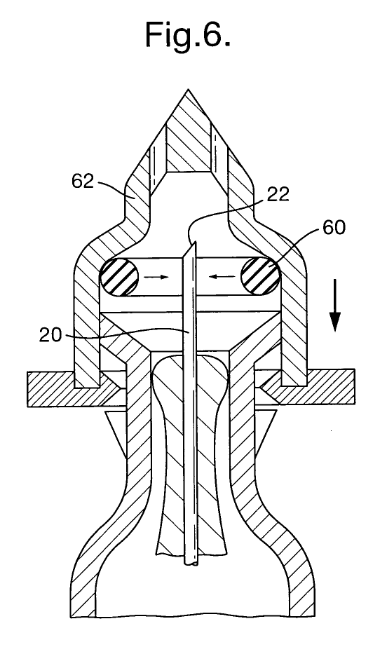

FIG. 6 illustrates an alternative arrangement of a puncture member;

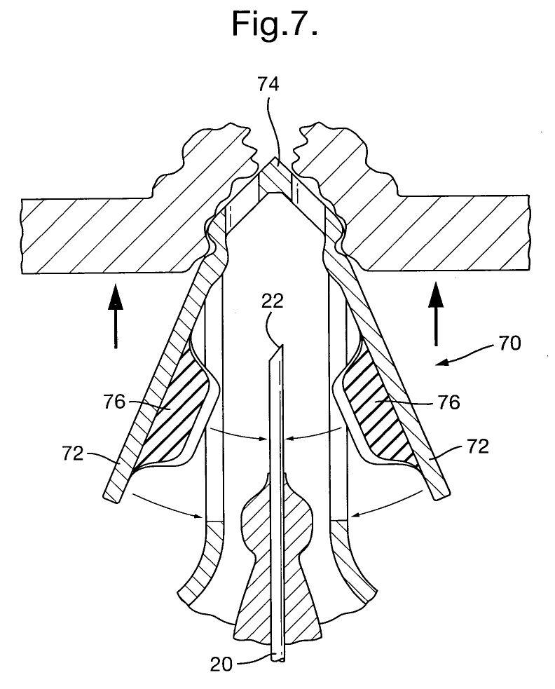

FIG. 7 illustrates a further alternative arrangement of a puncture member;

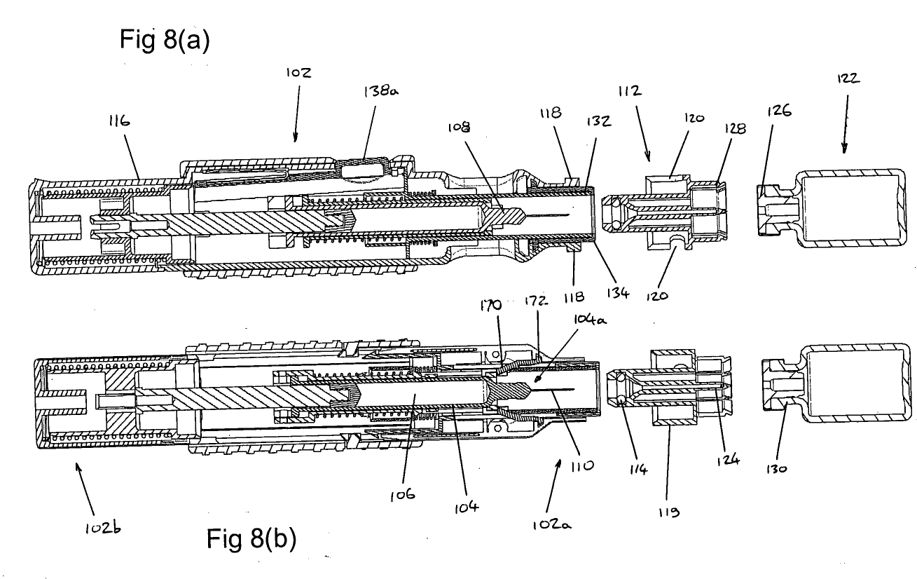

FIGS. 8(a) and (b) illustrate mutually perpendicular cross-sections through an auto injector embodying the present invention, together with an adaptor and a vial;

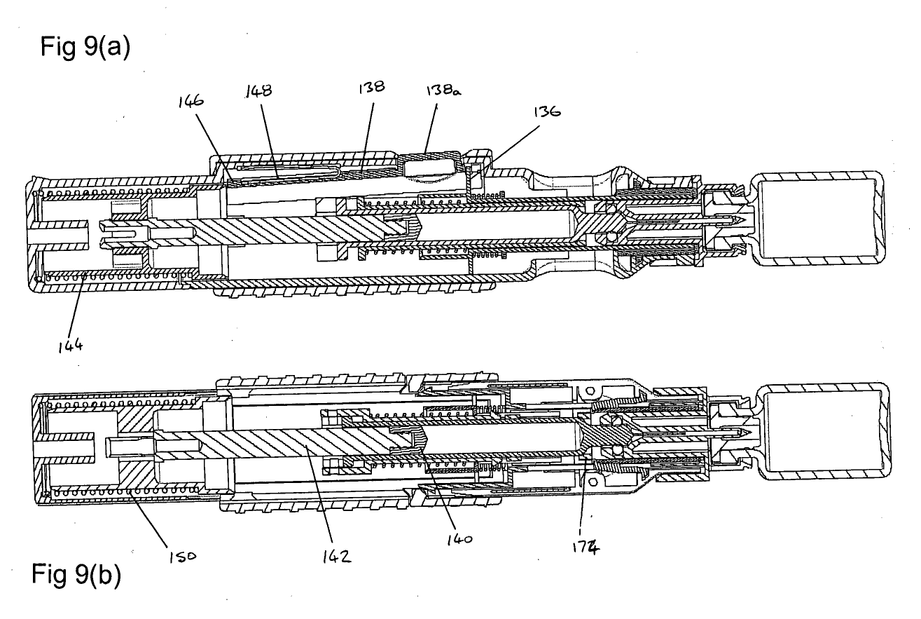

FIGS. 9(a) and (b) illustrate the components of FIGS. 8(a) and (b) in an assembled condition;

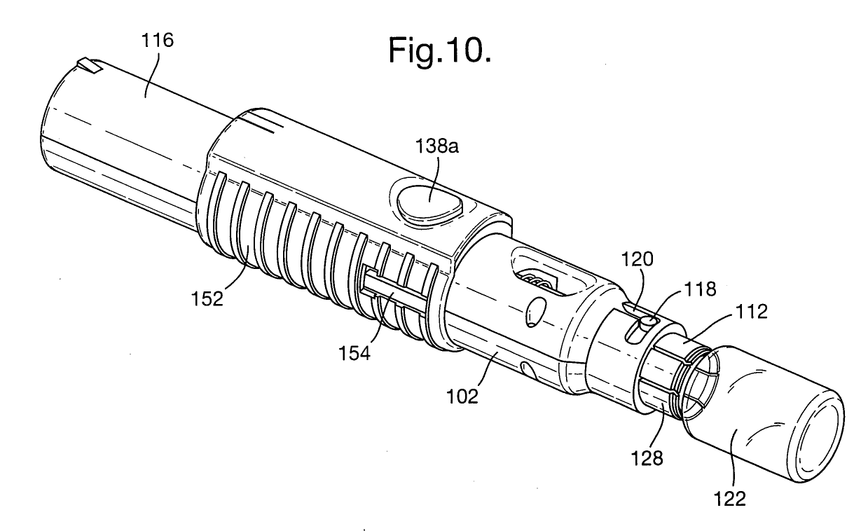

FIG. 10 illustrates the arrangement of FIGS. 9(a) and (b) as a perspective view;

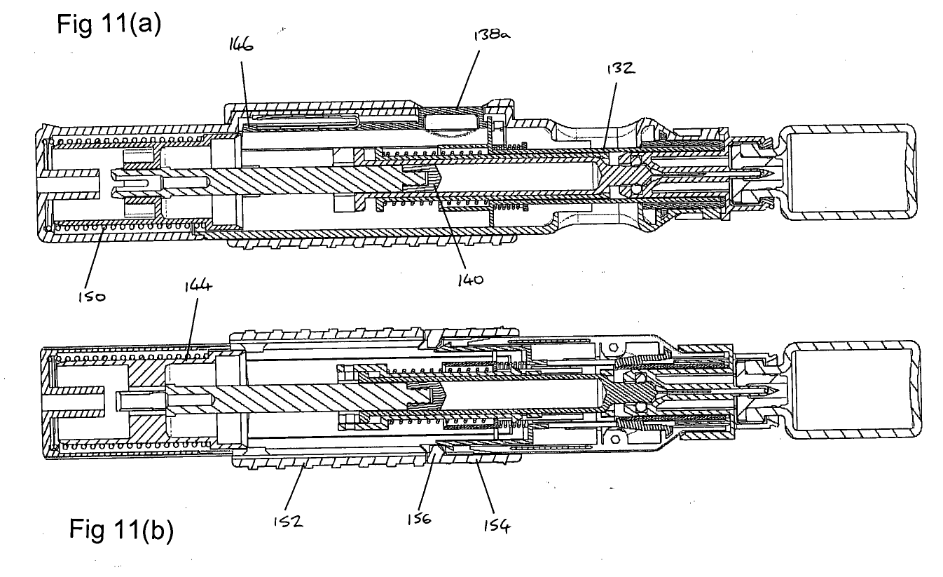

FIGS. 11(a) and (b) illustrate the arrangement of FIGS. 9(a) and (b) actuated to expel the pre-filled liquid;

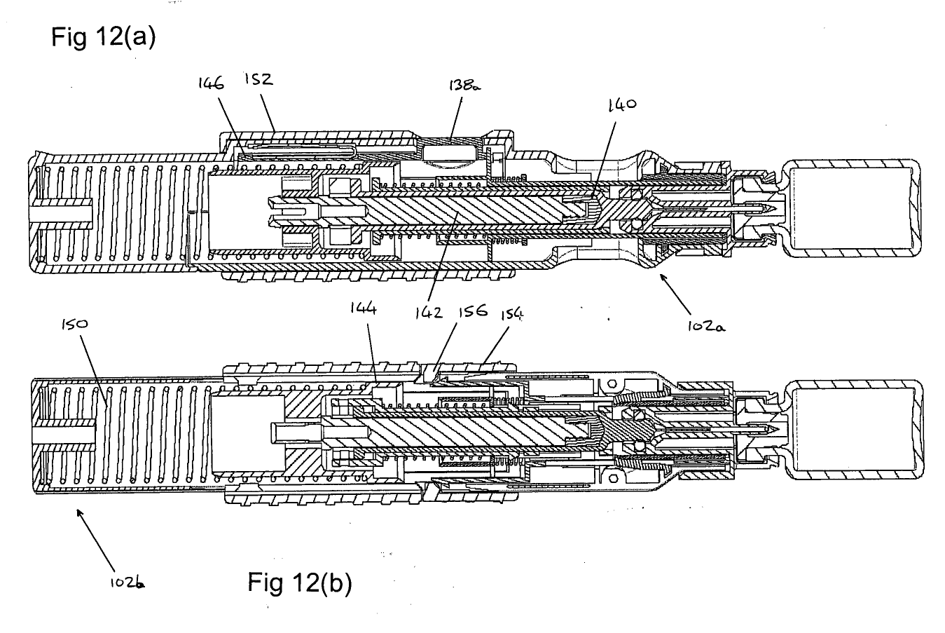

FIGS. 12(a) and (b) illustrate the arrangement having expelled the pre-filled liquid into the vial;

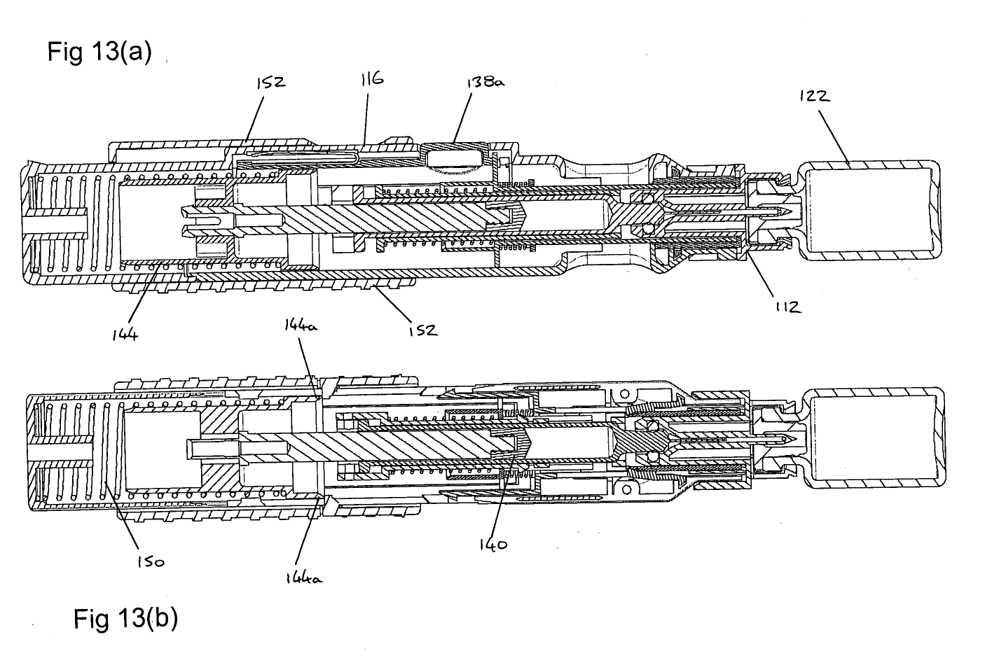

FIGS. 13(a) and (b) illustrate the arrangement during the process of drawing medicament into the auto injector from the vial;

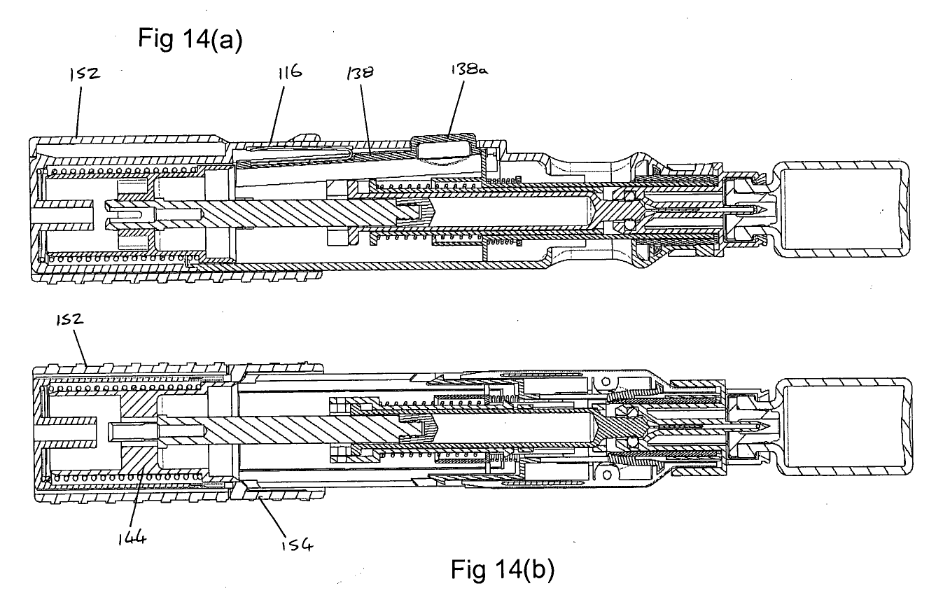

FIGS. 14(a) and (b) illustrate the arrangement containing medicament and ready for deployment;

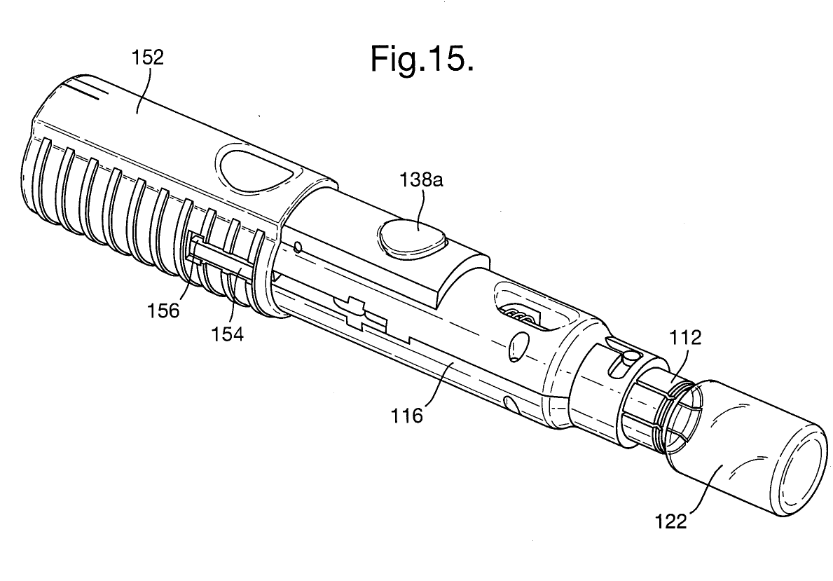

FIG. 15 illustrates the arrangement of FIGS. 14(a) and (b) as a perspective view;

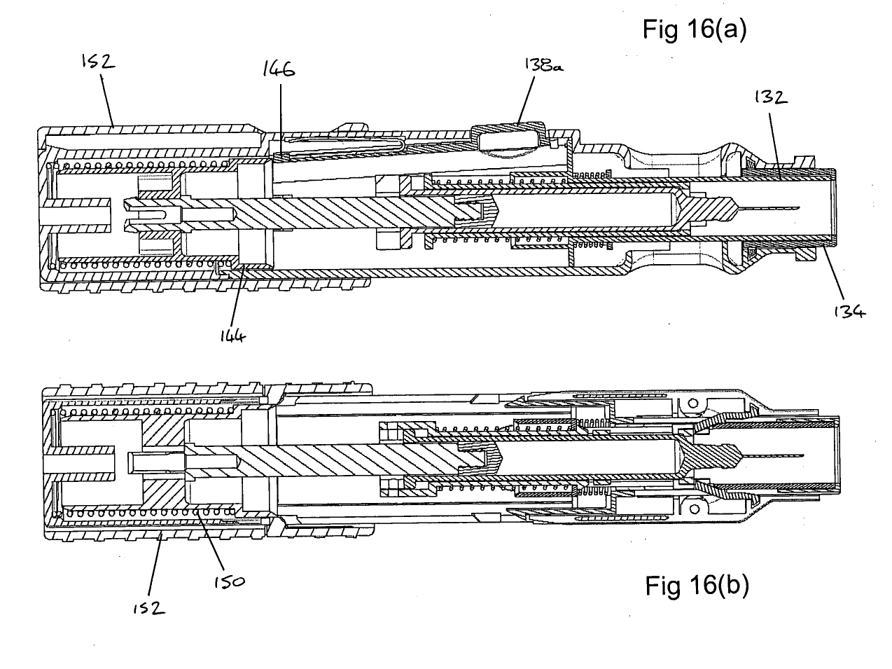

FIGS. 16(a) and (b) illustrate the auto injector ready for use;

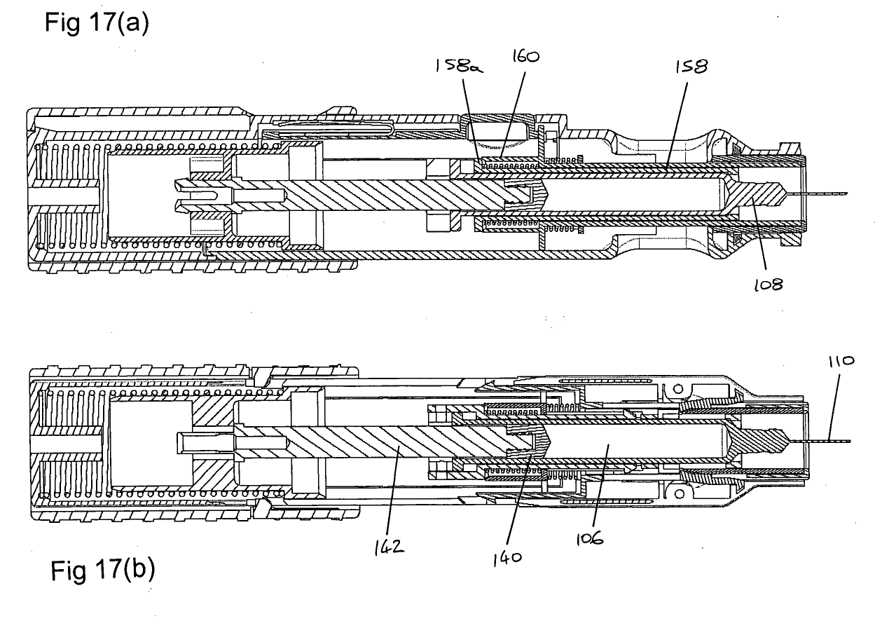

FIGS. 17(a) and (b) illustrate the auto injector with the syringe body deployed in the injection direction;

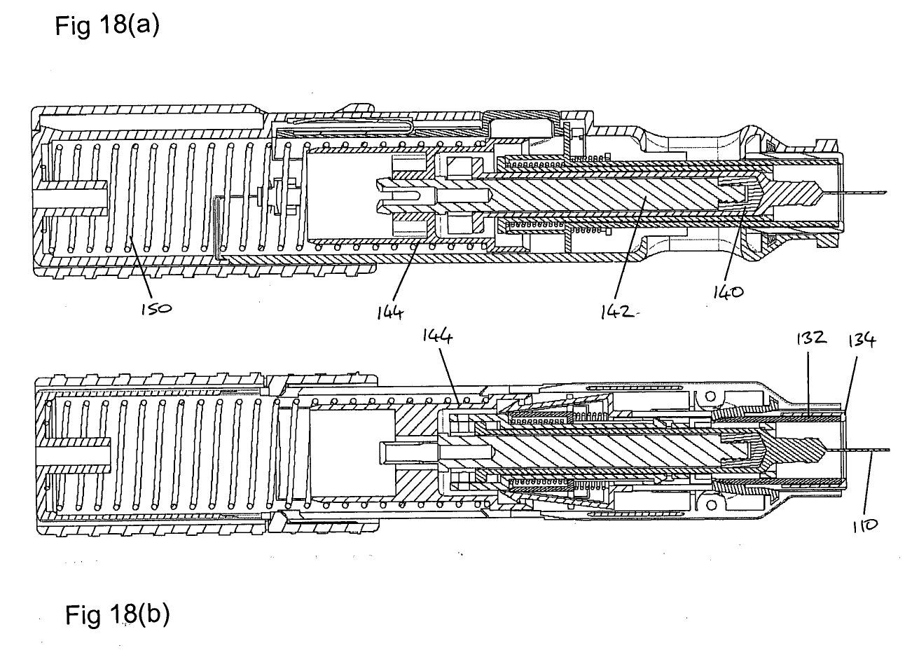

FIGS. 18(a) and (b) illustrate the auto injector having ejected the previously contained medicament;

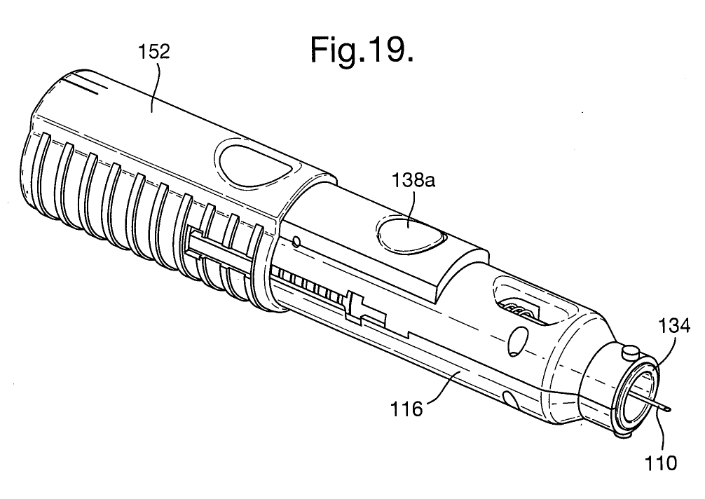

FIG. 19 illustrates the arrangement of FIGS. 18(a) and (b) as a perspective view;

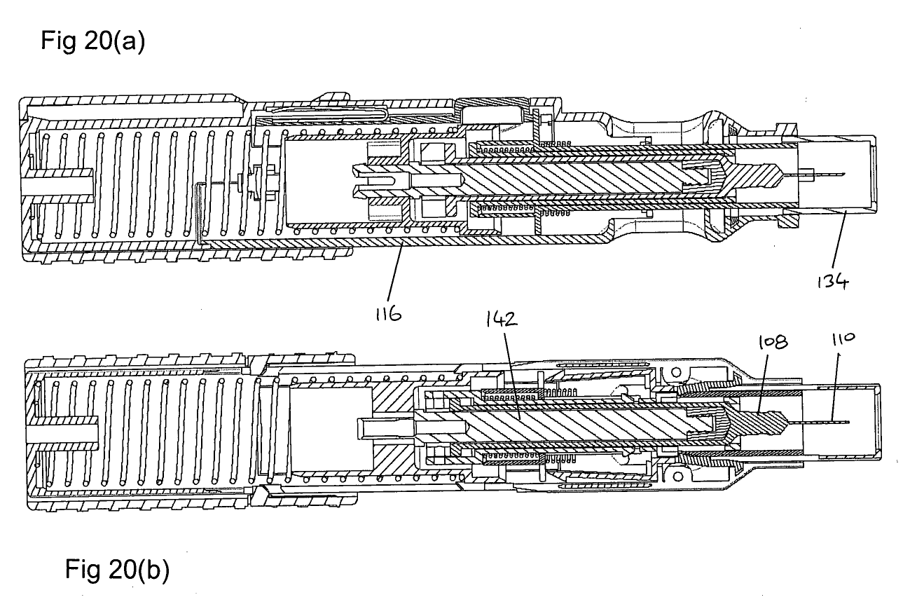

FIGS. 20(a) and (b) illustrate the auto injector with an outer collar extended to cover the needle on retrieval from a patient site;

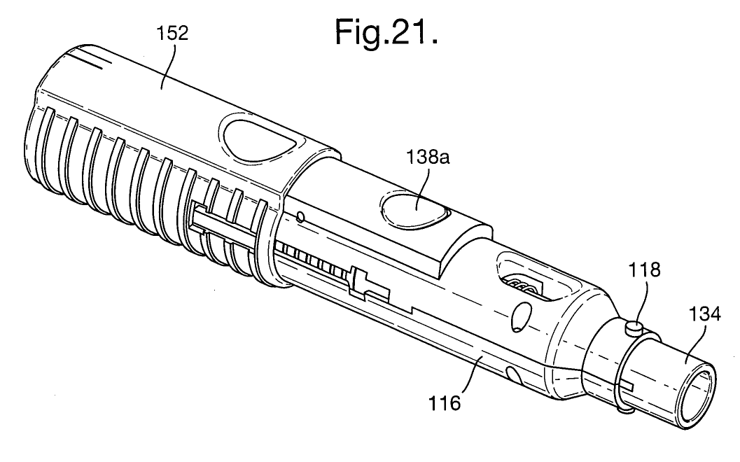

FIG. 21 illustrates the arrangement of FIGS. 20(a) and (b) as a perspective view;

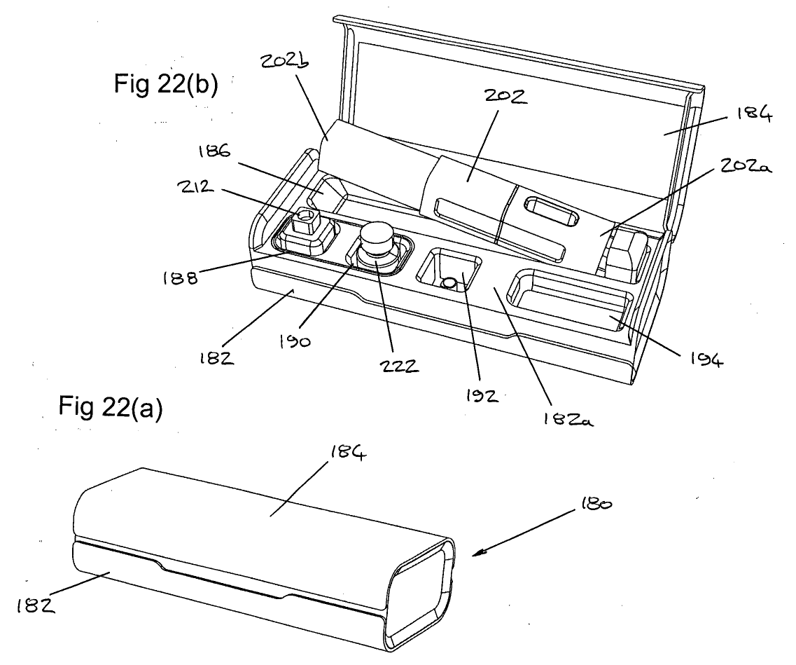

FIG. 22(a) illustrates a casing for an auto injector, adaptor and vial;

FIG. 22(b) illustrates an auto injector, adaptor and vial in the casing of FIG. 22(a);

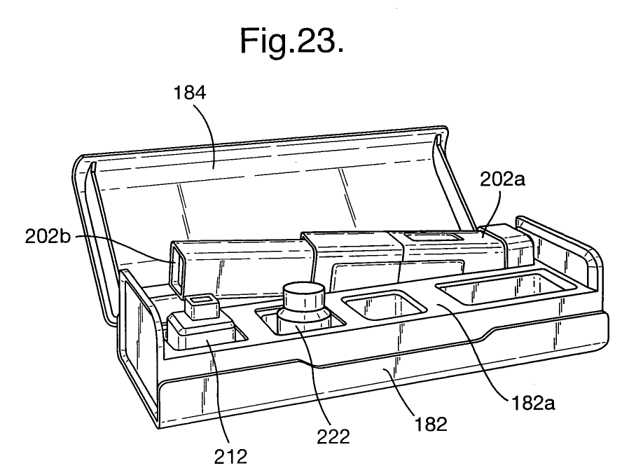

FIG. 23 illustrates a similar arrangement to that of FIG. 22(b);

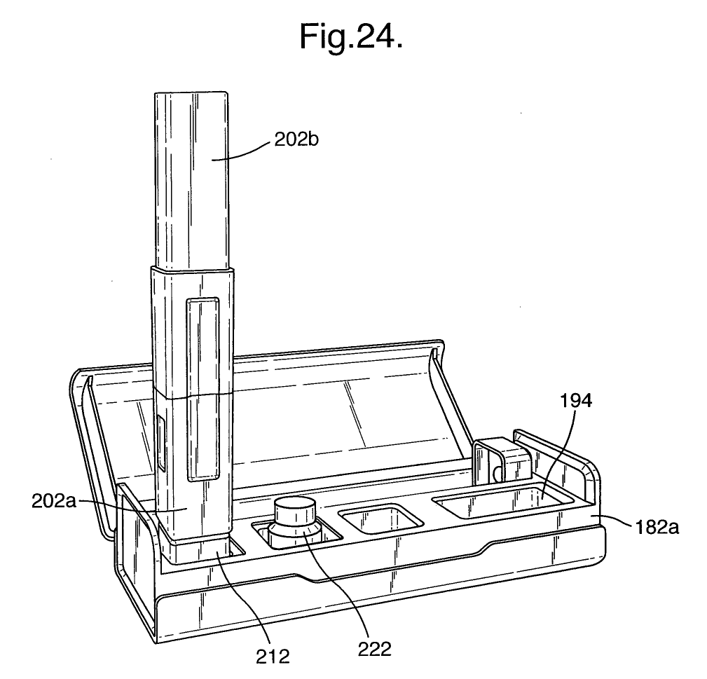

FIG. 24 illustrates the auto injector attached to the adaptor in the casing;

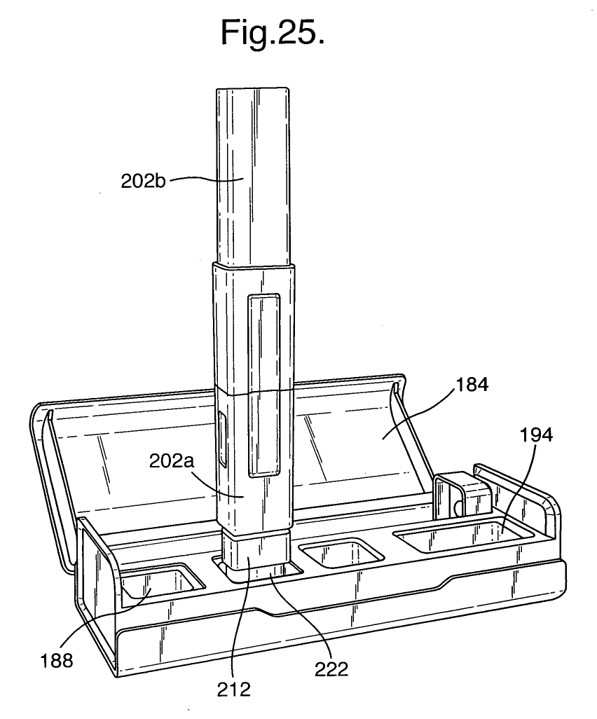

FIG. 25 illustrates the auto injector and adaptor attached to the vial in the casing;

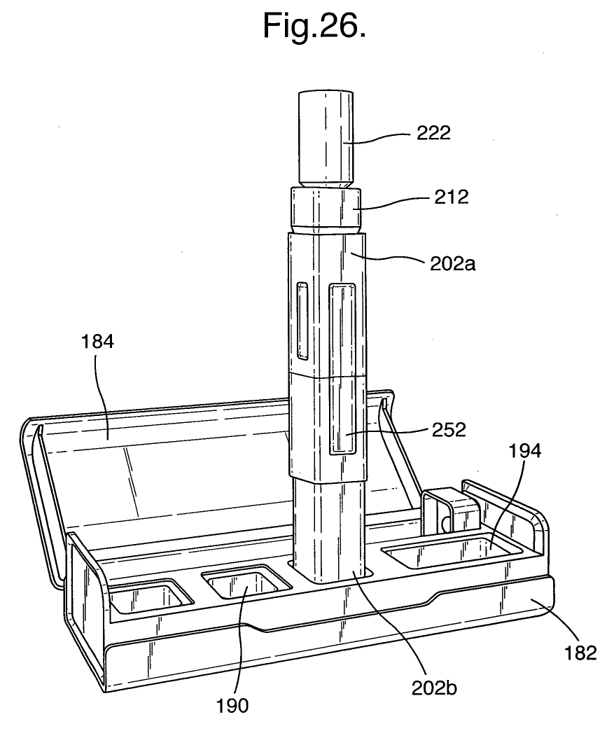

FIG. 26 illustrates the auto injector, adaptor and vial inverted and mounted in the casing ready to move fluid into the vial;

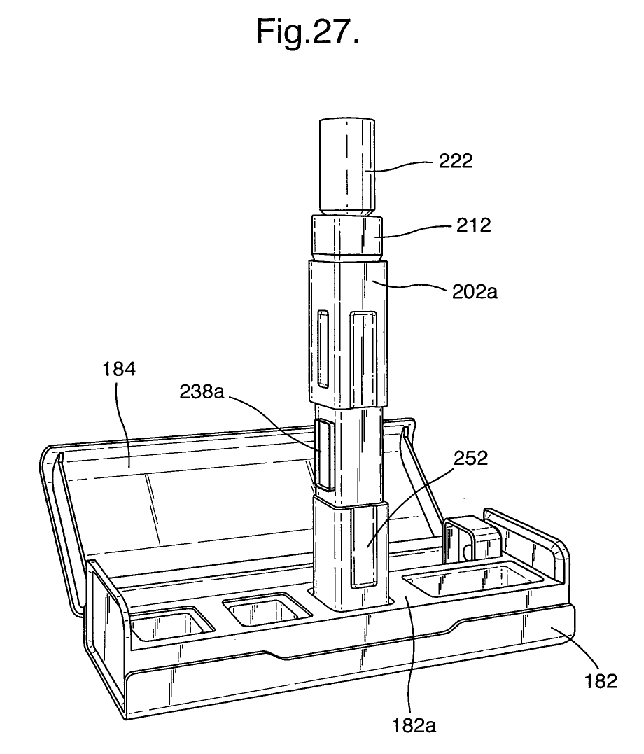

FIG. 27 illustrates the arrangement of FIG. 26 with a slider moved to draw medicament from the vial into the auto injector;

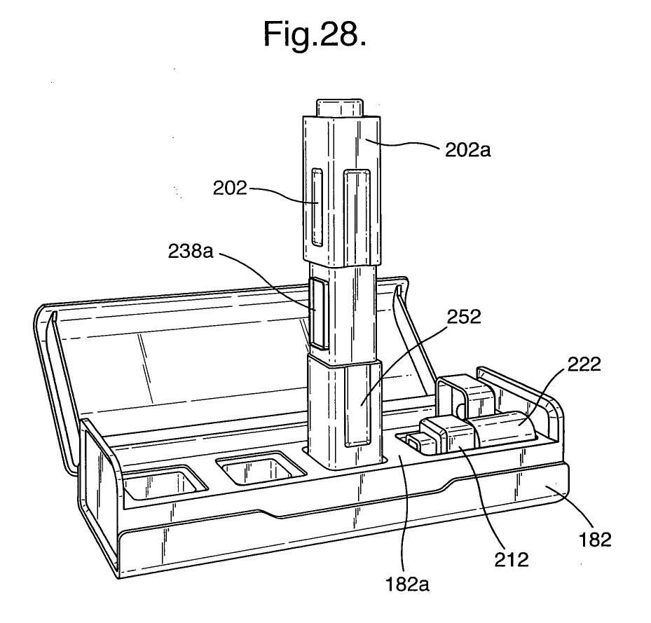

FIG. 28 illustrates the adaptor and vial assembly removed from the auto injector and stored in the casing; and

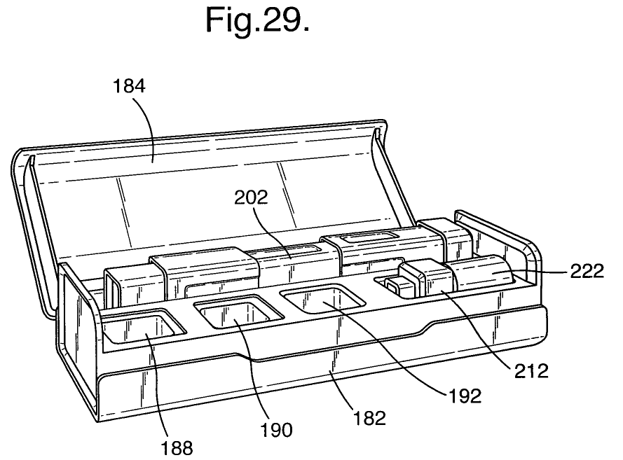

FIG. 29 illustrates the auto injector returned to the casing after use, ready to be closed and disposed of.

The following description includes details of various embodiments relating to an auto injector which may be pre-filled with a fluid, such as water or a buffer, for use in forming a medicament for subsequent dispensing using the auto injector. In this respect, FIG. 10 is a view of an auto injector set including an auto injector and adaptor, together also with a vial with which the auto injector set can be used. In some embodiments, the auto injector need not be prefilled with fluid but instead used only to draw in a medicament prior to deployment.

Before considering the details of this preferred embodiment and other arrangements, a description will be provided of an adaptor for use with a syringe. Many features of the syringe adaptor are relevant and can be applied to the auto injector adaptor, though it should be appreciated that the auto injector adaptor can be configured to fit with the housing of the auto injector, as well as seal with the syringe body contained within the auto injector and forming the container for the medicament to be dispensed.

As illustrated in FIG. 1, an adaptor 2 may be fitted to the body 4 of a syringe 6. The adaptor 2 is then also fitted to or engages with a vial 8 by means of an attachment member 10.

FIG. 2 illustrates the arrangement of FIG. 1 in cross-section.

The syringe 6 is of standard construction and includes a cylindrical elongate body 4 having internal walls 12 defining an internal cylinder or cylindrical space 14 for containing a fluid. In the example to be described, the syringe 6 is provided pre-filled with (sterile) water. In a standard way, a piston 16 is provided in the cylinder 14 and is movable along the inner walls 12 so as dispense fluid from the syringe 6 or to draw fluid into the syringe 6.

At a distal end, a finger flange 18 is provided in a standard maimer. Although not illustrated, an elongate drive component may be provided which extends from the piston 16 out of the syringe body 4, past the finger flange 18 so as to allow operation of the piston 16.

At the opposite end of the syringe 6, described here as the injection end, a needle 20 is provided which extends axially from the syringe body 4 to a needle tip 22. This part of the arrangement is illustrated more clearly in the enlarged portion of FIG. 2 which is illustrated in FIG. 3.

In a standard manner, the body 4 of the syringe 6 is provided with a neck portion 24 between the cylinder 14 of the body 4 and the needle 20. Although not illustrated, a passageway is provided in the neck portion 24 connecting the cylinder 14 to the needle 20. In the normal manner, the needle 20 is a hollow shaft which provides fluid communication between the needle tip 22 and the passageway in the neck portion 24.

As illustrated, the adaptor 2 includes a housing 26 which is mounted onto the syringe 6.

As illustrated, the housing 26 includes internal walls 28 which are located around the periphery of the neck portion 24 such that a cavity 30 is defined therebetween.

It would be possible for the housing 26 to seal with a portion of the shaft of the needle 20 or with the body 4 of the syringe 6. However, as illustrated, the housing 26 is arranged to seal with the neck portion 24 of the syringe 6.

As illustrated, an inwardly facing O-ring 32 is provided for sealing with the neck portion 24. In particular, an annular recess 34 is provided in the inner wall 28 of the housing 26 for receiving the O-ring. The O-ring 32 then seals with an outer surface of the neck portion 24. Of course other seals could be provided such as a comoulded seal or lip seal.

The adaptor 2 is also provided with what will be described as a puncture member 36 which extends away from the housing 26 of the adaptor 2 and the syringe 6. The puncture member 36 is arranged to puncture a septum 38 of a vial 8. In particular, the puncture member 36 is provided with a puncture portion 40 at its end furthest from the housing 26. The puncture portion 40 is preferably pointed in some way so as easily to puncture the septum 38 of the vial 8. Ports 42 are provided in the outer surface 44 of the puncture member 36 and connect to a communication channel 46 defined by an internal surface 48 in the puncture member 36. Thus, the communication channel 46 provides fluid communication between the ports 42 and the cavity 30.

Notably, the puncture member 36 and housing 26 are arranged such that the needle tip 22 of the needle 20 does not contact either the inner surfaces 48 of the puncture member 36 or the inner surfaces 28 of the housing 26, even during installation of the syringe 6 with the adaptor 2. Thus, as long as the syringe 6 remains mounted to the adaptor 2, the needle tip 22 remains protected and can subsequently be used by a user for injection in a patient. The puncture member 36 allows the septum of a vial 8 to be punctured and to provide fluid communication to the cylinder 14 of the syringe 6 by means of the ports 42.

In some arrangements, the adaptor 2 may be arranged such that the needle 20 extends only into the cavity 30 of the housing 36 and not into the communication channel 46. As long as fluid communication is provided between the communication channel 46 and the cavity 30, the puncture member may then be positioned and orientated on the adaptor in any fashion. However, as illustrated, efforts are taken to reduce volume of the cavity 30 and the volume in the communication channel 46 as these volumes are effectively “dead” and will contain fluid that will merely be lost.

Thus, as illustrated, the communication channel 46 is provided coaxially with the cavity 30 such that the cavity 30 receives the neck portion 24 of the syringe 6 and the communication channel 46 receives the needle 20 along substantially the entire length of the communication channel 46.

As illustrated, the housing 26 and puncture member 36 are arranged such that, subject to practical constraints of construction, the inner surface 48 of the puncture member is as close as possible to the outer surface of the shaft 20 of the needle and the inner surface of the housing 28 is as close as possible to the outer surface of the syringe body 4, in particular the neck portion 24 of the syringe body 4 as illustrated. In this way, losses of fluid or medicament may be minimised.

Before considering operation of the adaptor 2 some description is provided of two preferred features, namely, provision of a support member 50 for supporting the adaptor 2 relative to the syringe 6 and the attachment member 10 mentioned above.

As illustrated, so as to support the adaptor 2 relative to the syringe 6 when the adaptor 2 is mounted on and sealed with the syringe 6, a support member 50 is provided which extends beyond the seal towards the finger flange 18 end of the syringe 6 so as to engage with the outer surface of the body 4 of the syringe 6. Preferably, the support member 50 comprises a number of elongate longitudinally or axially extending arms for gripping the exterior of the body 4 of the syringe 6. More preferably, the support member 50 is a cylindrical extension of the housing 26 having an internal diameter arranged to receive and support the outer surface of the elongate body 4 of the syringe 6.

In this way, when the housing 26 is sealed to the syringe 6, the adaptor is held securely relative to the syringe 6, in particular preventing tilting of the adaptor 2 relative to the axis of the syringe 6.

The attachment member 10 extends away from the housing 26 in a direction away from the syringe 6 and substantially parallel with the puncture member 36.

The illustrated vial 8 is of a standard construction and includes a neck 52. As illustrated, the attachment member 10 is arranged to engage with the neck 52 of the vial 8. Preferably, the attachment member 10 extends on opposite sides, or even in a circumferential arrangement, of the puncture member 36 so as to engage with opposite sides of the neck 52 of the vial 8. In the illustrated embodiment, the attachment member 10 comprises a plurality of elongate legs which together form a substantially annular body surrounding the neck 52 of the vial 8. The elongate legs have some resilience or at least flexibility allowing them to deflect in a radially outward direction. Thus, as the adaptor 2 is pushed onto the vial 8 so that the puncture member 36 punctures the septum 38 of the vial 8, the legs of the attachment member 10 are deflected outwardly around the end of the vial 8 and then subsequently engage with the neck 52 of the vial 8 so as to secure the vial 8 in place with the puncture member 36 and its ports 42 inside the vial 8.

As illustrated, the attachment member is arranged to form a latch such that the attachment member 10 non-releasably engages with the neck 52 of the vial 8. In particular, as illustrated, the legs of the attachment member 10 are sloped gently outwardly in a direction such that when the end of the vial 8 is inserted between the legs of the attachment member 10, those legs deflect outwardly around the end of the vial 8. However, the legs are sloped more steeply in the opposite direction such that once the legs of the attachment member 10 have engaged with the neck 52 of the vial 8, it becomes very difficult to remove the end of the vial 8 from the attachment member 10 of the adaptor 2.

There will now be given a description of use of the adaptor 2 in an embodiment where a syringe 6 is provided pre-filled with water for mixing with a lyophilised medicament provided in a vial 8.

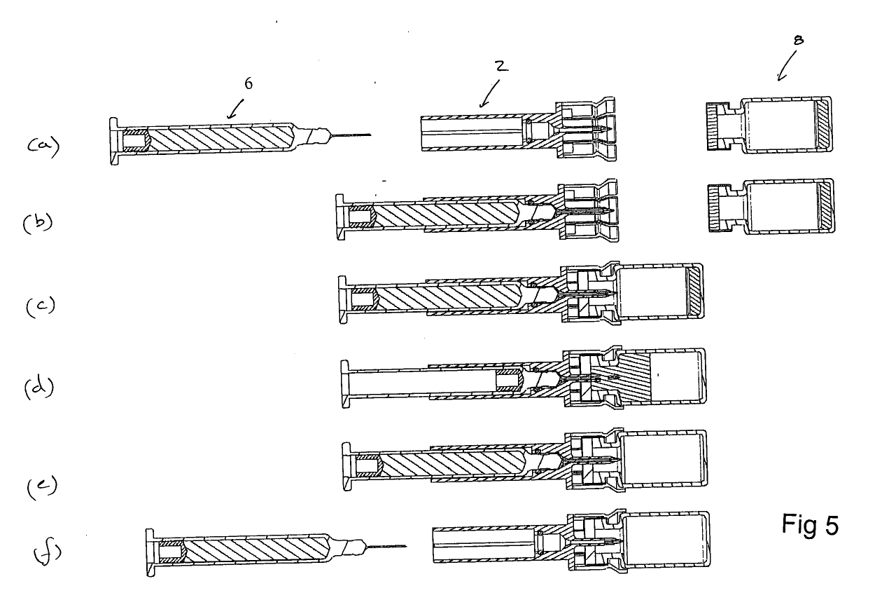

FIGS. 4(a) and 5(a) illustrate the syringe 6, adaptor 2 and a vial 8 before assembly.

First, the syringe 6 is fitted into the adaptor 2 as illustrated in FIGS. 4(b) and 5(b). The housing 26 of the adaptor 2 seals with the syringe 6 and the needle 20 extends inside the communication channel 46 of the puncture member 36. It is envisaged that adaptors might be sold separately, but also that assemblies of a syringe with a fitted adaptor might also be sold.

As illustrated in FIGS. 4(c) and 5(c), the assembly of the syringe 6 and adaptor 2 may then be fitted to a vial 8. In particular, the septum end of the vial 8 is pushed between the legs of the attachment member 10 of the adaptor 2 such that the puncture portion 40 of the puncture member 36 punctures and penetrates the septum 38 of the vial 8 and the legs of the attachment member 10 engage with the neck 52 of the vial 8. Once in this configuration, the piston 16 may be driven along the cylinder 14 of the syringe 6 so as to expel the contents of water through the needle 20, cavity 30, communication channel 46 and ports 42 into the inside of the vial 8. This is illustrated in FIGS. 4(d) and 5(d).

After the water and lyophilised medicament in the vial 8 have been mixed, and with the vial 8 uppermost, the piston 16 may then be withdrawn along the cylinder 14 of the syringe 6 so as to draw the mixed fluid back through the ports 42, communication channel 46, cavity 30 and needle 20 into the cylinder 14. This is illustrated in FIG. 4(e) and 5(e).

A user may then pull the syringe 6 away from the adaptor 2 and vial 8, thereby exposing the needle 20 and needle tip 22 for the first time as illustrated in FIGS. 4(f) and 5(f). By means of the latch provided by the legs of the attachment member 10, the adaptor 2 is held securely attached to the vial 8.

Although not illustrated, the adaptor 2 may additionally be provided with a shield which fits between the legs of the attachment member 10 so as to cover and protect the puncture member 36. In particular, the shield preferably covers and protects the ports 42 so as to maintain the needle 20 in a sterile environment. Use of the shield may be particularly advantageous when the assembly of syringe 6 and adaptor 2 illustrated in FIGS. 4(b) and 5(b) is sold as a single unit. A user receives the assembly of the syringe 6 and adaptor 2 with a shield fitted. The shield is then removed from the attachment member before the adaptor is then fitted to a vial.

FIG. 6 illustrates an alternative construction for the puncture member whereby sealing occurs with the outer surface of the shaft of the syringe needle, in addition to or rather than with the body of the syringe. In this respect, the puncture member can itself be considered to be part of the housing, the cavity being formed between the puncture member and the needle.

As illustrated in FIG. 6, the adapter is two separate pieces that slide together. The first piece is in direct contact with the syringe body and the second piece pierces the vial septum.

As illustrated in FIG. 6, an O-ring 60 is provided within the puncture member 62. When the puncture member 62 is pressed against the septum of a vial to puncture the vial, the puncture member 62 reacts or is forced backwards so as to move the O-ring 60 inwardly and seal against the outer surface of the shaft of the needle 20. The two sections of the adapter move towards one another until they are (unreleasably) locked together to hold the O-ring seal in contact with the needle. Because the O-ring is in its expanded state when the needle 20 is fitted inside the puncture member 62, there is no danger of the tip 22 of the needle 20 contacting inner surfaces of the adaptor. This adapter can be designed so that the force required to pierce the septum exceeds that required to lock the adapter so that the adapter reaches its locked state before the vial is pierced. Withdrawing the needle with the O-ring in contact will not blunt the needle tip due to the direction of travel.

FIG. 7 illustrates another alternative arrangement of a puncture member where sealing between the adaptor and the syringe again occurs on an outer surface of the needle 20. In this arrangement, the puncture member 70 includes at least two opposing legs 72 which are hinged at a position proximate the puncture portion 74. As the puncture portion 74 is pushed through the septum of a vial, the septum of the vial acts on the legs 72 so as to push them inwardly in a radial direction. The legs 72 could have compliant material to achieve seal with needle. The legs 72 are provided with seals 76 for sealing against the outer surface of the shaft of the needle 20. Thus, with the puncture member 70 in its inserted position through the septum of the vial, the legs 72 are held inwardly so that the seals 76 seal against the needle 20 and define the required cavity. Where only two legs 72 are provided, then each seal 76 makes up half of the complete seal around the needle 20. However, it will be appreciated that three or more legs could be provided, each with their proportionate amount of seal. As with the example of FIG. 6, during insertion of the needle 20 into the puncture member 70, there is no danger of inner surfaces of the puncture member 70 contacting the needle tip 22 during assembly. Furthermore, lost volume is significantly reduced. Indeed, if an appropriate seal is present elsewhere, the legs of this arrangement could be used merely to reduce the lost volume.

Another method for closing out the dead volume is to expand/contract flexible sections of the adapter or by advancing a ring-like part of the adapter distally to close out volume.

A description is now provided of an auto injector set embodying the present invention, the auto injector set including an auto injector 102 embodying the present invention, together with an adaptor 112 embodying the present invention and for use with that auto injector. FIGS. 8(a) and (b) illustrate the auto injector set, together with a vial 122. In particular, FIGS. 8(a) and (b) are both cross-sections of the three components taken along their common axis with the cross-section of FIG. 8(a) being perpendicular to the cross-section of FIG. 8(b).

The auto injector 102 includes a syringe body 104 forming a container for storing a volume of fluid 106, such as sterile water. At an ejection end 104a of the syringe body 104, an outlet portion 108 is formed as part of the neck of the syringe body 104. The outlet portion 108 includes a needle 110.

As illustrated in FIGS. 9(a) and (b), the adaptor 112 is fitted to the end of the auto injector 102 so as to seal with the outlet portion 108 of the syringe body 104. In particular, in the illustrated embodiment, an O-ring 114 is provided to seal with the neck of the syringe body 104 forming part of the outlet portion 108. Of course, it should be understood that the adaptor 112 could alternatively be formed in the manner of any of the adaptors described above. However, unlike the adaptors described above, the adaptor 112 does not need to fit to the syringe body 104 for support, but engages with the housing 116 of the auto injector 102.

In general, the adaptor 112 includes a mounting portion 119 in the form of a collar which fits around the injection end 102a of the housing 116 of the auto injector 102. The injection end 102a can be considered as a receiving portion on which the mounting portion 119 of the adaptor 112 is fitted. The mounting portion 119 thus provides physical support to the adaptor 112 and secures it in place. In preferred embodiments, the mounting portion prevents axial movement between the adaptor 112 and the auto injector 102. This may be achieved by means of a threaded or bayonet fitting. The illustrated embodiment uses a bayonet fitting.

In particular, in the illustrated embodiment, a pair of diametrically opposed pins 118 are provided on an exterior surface of the housing 116 at the injection end 102a of the auto injector 102. An outer portion of the adaptor 112 is formed with corresponding L-shaped slots 120 so as to form a bayonet fitting. This is also illustrated in FIG. 10.

In a manner similar to that described above, the adaptor 112 can also be fitted to the vial 122, for instance as illustrated in FIGS. 9(a), 9(b) and 10. The puncture member 124 of the adaptor punctures the septum 126 of the vial 122 and legs of an attachment member 128 of the adaptor 112 engage with and securely hold the neck 130 of the vial 122.

In a preferred method of assembly to be described below, the adaptor 112 is first fitted to the auto injector 102 and then the auto injector set formed by the auto injector 102 and adaptor 112 is fitted to the vial 122. However, it should be appreciated that it would also be possible to fit the adaptor 112 to the vial 122 before fitting the adaptor 112 to the auto injector 102.

As illustrated in FIGS. 8(a) and (b), the auto injector 102 includes an inner collar 132 and an outer collar 134 which extend axially at the injection end 102a of the auto injector 102. These two collars are slidable axially of the auto injector 102 and preferably biassed outwardly of the housing 116. As will be discussed below, these collars 132, 134 can be pushed inwardly of the auto injector 102 by being pressed against a patient's skin. However, as illustrated in FIGS. 9(a) and (b), these collars 132, 134 can also be pushed inwardly of the auto injector 102 by the adaptor 112.

The inner collar 132 includes, at an inner end, a release member 136 which, as illustrated, takes the form of a flange.

A button 138a of an actuator 138 extends through an aperture defined in the walls of housing 112 of the auto injector 102. The release member 136 and actuator 138 are arranged to interact such that, with the inner collar 132 in its extended position as illustrated in FIGS. 8(a) and (b) and the release member 136 to the right as illustrated, the actuator 138 cannot be moved. In contrast, with the inner collar 132 pressed into the housing 116 of the auto injector 102 and the release member 136 to the left position as illustrated in FIGS. 9(a) and (b), the button 138a of the actuator 138 can be depressed radially inwardly of the auto injector 102.

As illustrated, a piston 140 is provided in the bore of the syringe body 104. The piston 140 is slidable along the walls of the bore of the syringe body 104 and seals with those walls. Hence, sliding the piston 140 towards the outlet portion 108 and injection end 102a of the auto injector 102 will cause the fluid 106 in the syringe body 104 to be ejected through the adaptor 112 and into the vial 122. A plunger rod 142 connects the piston 140 to a main drive element 144. As illustrated in FIGS. 9(a) and (b) the main drive element 144 is held in place at the far actuation end 102b of the auto injector 102 by means of an operating end 146 of the actuator 138.

As illustrated, the actuator 138 is pivotable about a midpoint such that depression of the button 138a of the actuator 138 inwardly of the auto injector 102 causes the operating end 146 of the actuator 138 to move outwardly and to disengage from the main drive element 144 as illustrated in FIGS. 11(a) and (b). A spring, such as leaf spring 148 may be provided to bias the actuator 138 into a position for engaging with the main drive element 144.

A main drive spring 150 is provided between an inner end wall of the housing 116 of the auto injector 102 and a flange on the main drive element 144. The main drive spring 150 is provided initially in a compressed state. When the operating end 146 of the actuator 138 disengages from the main drive element 144, the main drive element 144 is driven by the main drive spring 150 towards the injection end 102a of the auto injector 102 and, hence moves the plunger rod 142 and piston 140 towards the injection end 102a of the auto injector 102 as illustrated in FIGS. 12(a) and (b) so as to eject the fluid 106 stored in the syringe body 104 into the vial 122. For completeness, it will be noted that the adaptor 112 prevents the syringe body 104 itself from being moved towards the injection end 102a of the auto injector 102.

The actuator 138, main drive element 144 and main drive spring 150 can be considered as the first part of a filling mechanism used first to expel water from the auto injector 102 and then to re-fill the auto injector 102 with a medicament ready for dispensing. The actuator 138 may thus be considered as a user actuatable filling actuator.

The filling mechanism also includes an outer sleeve 152 slidable axially along the housing 116 of the auto injector 102. In the illustrated embodiment, the outer sleeve 152 includes walls defining an aperture through which the button 138a of the actuator 138 extends. However, as most clearly illustrated in FIG. 12(a) when the auto injector 102 has ejected the fluid into the vial 122 and the piston 140 has been moved to the outlet portion 108 of the syringe body 104, the actuator 138 is retained in its actuation position with the button 138a inwardly of the auto injector 102. Hence, the actuator 138 and its button 138a are positioned inwardly of the outer sleeve 152 and allow the outer sleeve 152 to be slid along the housing 116.

The outer sleeve 152 as illustrated includes a pair of resilient arms 154 with inwardly extending engagement portions 156 which, as best illustrated in FIG. 13(b) engage with the main drive element 144. As illustrated, they engage with the injection end 144a of the main drive element 144. Thus, by manually moving the outer sleeve 152 along the length of the auto injector 102 away from the injection end 102a and towards the actuation end 102b, the outer sleeve 152 moves the main drive element 144 back to its original position and re-compresses the main drive spring 150 so as to replenish the energy/power of that spring 150. Of course, by virtue of this movement, the plunger rod 142 moves the piston 140 along the bore of the syringe body 104 away from the outlet portion 108 so as to draw into the container formed by the syringe body 104 the mixed medicament formed in the vial 122. To ensure that the mixed medicament in the vial 122 is correctly drawn through the outlet portion 108, it is usually preferable that the actuation and 102b is placed on a surface with the vial 122 above the auto injector 102. At the end of the travel, as illustrated in FIG. 14(a), the actuator 138 returns to its original position such that its operating end 146 again engages with the main drive element 144 to hold it in its initial state with the main drive spring 150 compressed. Hence, the auto injector 102 is placed in a state where medicament is contained within the auto injector 102 and is ready for dispensing.

FIG. 15 illustrates the auto injector 102 in this state before the adaptor 112 and vial 122 have been removed.

Having released the bayonet fitting between the adaptor 112 and the auto injector 102, the auto injector 102 is placed in the state as illustrated in FIGS. 16(a) and (b). As illustrated, the inner collar 132 and outer collar 134 again return to their extended positions (for instance under the power of a compression spring) and the release member 136 prevents actuation of actuator 138.

With the inner collar 132 and outer collar 134 pushed inwardly of the auto injector 102, for instance when pressed against a patient's body, the actuator 138 can be operated this time as a deployment actuator and, as illustrated in FIGS. 17(a) and (b) releases the main drive element 144. On this occasion, without the presence of the adaptor 112 at the injection end 102a of the auto injector 102, movement of the main drive element 144 and plunger rod 142 will first act to move the syringe body 104 and its outlet portion 108 towards the injection end 102a of the injection device 102 to the position as illustrated where the needle 110 extends by an appropriate length for penetration into a patient. In this respect, as illustrated, a support 158 for the syringe body 104 extends as a flange 158a to engage with a stopper 160 formed by part of the inner collar 132. Interaction of the flange 158a with the stopper 160 prevent the syringe body 104 from being moved any further towards the injection end 102a of the auto injector 102. Subsequent movement of the main drive element 144 and plunger rod 142 under the power of the main drive spring 150 will cause the piston 140 to move along the bore of the syringe body 104 towards the outlet portion 108 so as to expel the medicament contained in the syringe body 104 through the outlet portion 108 and needle 112. This state is illustrated in FIGS. 18(a) and (b) and FIG. 19.

In the illustrated embodiment, with the syringe body 104 and outlet portion 108 in the deployed position, the outer collar 134 is released and driven by means of a spring so as to extend further outwardly from the housing 116 of the auto injector 102 as illustrated in FIGS. 20(a) and (b) and FIG. 21. This provides a shield for the extended needle 110 and additional safety to the user. Whenever inner collar 132 is not depressed by the adaptor 112 or pressed against a patient, the legs 170 of syringe stop 172 are in their normal position. However, when the auto injector is ready for use, either with the adaptor 112 fitted or against a patient, the legs 170 are ramped out of the way of syringe holder 174 by the inner collar 132 allowing the syringe body 104 to move forward. This prevents the syringe body 104 moving forward when the needle cover is removed and the device is dropped.

The auto injector arrangement described above uses the same actuator for actuating the filling mechanism to expel the initial contents 106 of the syringe body 104 into the vial 122 and for actuating the deployment mechanism to deploy the syringe body 104 and eject the medicament. However, embodiments are also possible using separate actuators. In one arrangement, the deployment actuator might be concealed by the outer sleeve until it has been slid back so as to refill the syringe body 104 with medicament. This would provide an additional safety feature preventing deployment of the auto injector 102 whilst it contained only the initial fluid.

Also, the above arrangement uses a spring to power the step of expelling the initial fluid and deploying/ejecting the medicament. Other arrangements are possible where a manual mechanism is provided for the user to operate the piston to expel the initial fluid from the syringe body 104. This could be provided in the manner of a slider similar to that described above. Indeed, the same slider could be used to first expel the initial contents of the syringe body 104 and then draw medicament back into it.

The above arrangement uses a manually operated refilling mechanism employing the slider 142. Alternatively, an internal power source, such as a spring could be provided to achieve this operation with the user merely having to actuate or release that power source.

It will be appreciated that power sources other than springs, for instance electrically powered, could be used. Also, it is not essential to use a sleeve which extends around the entire periphery of the housing 116 of the auto injector 102. Instead, other forms of slider might be used.

It should also be noted that similar auto injectors could be provided for use without an adaptor. In this case, as has been done previously with conventional syringes, a first needle or straw is used with the vial 122. This is then replaced with an injection needle prior to use of the auto injector.

In one preferred embodiment, an auto injector is provided as part of an auto injector pack in a casing, together with an adapter and a vial.

It should be noted that it would be possible to construct the auto injector such that the medicament container, for example the syringe body, could be removed after use and replaced with another container, either empty or pre-filled with fluid. In this way, the auto injector can be re-used.

FIG. 22(a) illustrates a casing 180 having a lower portion 182 and an upper portion 184, the upper portion 184 forming a cover for the lower portion 182.

As illustrated in FIG. 22(b), the lower portion 182 takes the form of a tray 182a which is shaped to receive and hold various components including an auto injector 202, an adapter 212 and a vial 222. In particular, the tray 182a of the lower portion 182 includes an auto injector receiving portion 186 shaped to receive the auto injector 202 with the axis of the auto injector 202 generally within the tray 182a. The tray 182a is also shaped to include an adapter receiving portion 188 and a vial receiving portion 190. The adapter receiving portion 188 and vial receiving portion 190 may be provided together in the same part of the lower portion 182 of the casing 180. This is advantageous in that it allows a seal, such as a foil to be placed over both receiving portions 188, 190 so as to seal the adapter 212 and 222 within their respective portions. The part including these two portions 188, 190 can then be sterilised, for instance using gamma radiation.

As illustrated, the tray 182a also includes a receiving portion 192 for the actuation end 202b of the auto injector 202 and also a receiving portion 194 for receiving the adapter 212 and vial 222 fitted together and positioned with their common axis generally within the tray 182a.

FIG. 22(b) also illustrates a preferred feature whereby, upon opening the upper portion or cover 184 of the case 180, the actuator end 202b of the injection device 202 is caused to be raised up out of the tray 182a of the lower portion 182. The significance of this will become apparent from the following description.

To raise the actuator end 202b up out of the tray 182a, the receiving portion 186 could include a resilient member, such as a spring or a block of resilient material, for instance a foamed material, which expands so as to lift the actuator end 202b. Alternatively, some mechanism linked to opening the upper portion 184 could be provided. For example, a strap could be fixed at one end to the lower portion 182, extend under the actuation end 202b and be fixed at the opposite end to the upper portion 184. Upon opening the upper portion 184, the strap is pulled and lifts the actuation end 202b.

Starting from the arrangement illustrated in FIG. 23 with the upper portion or cover 184 of the casing 180 open, a user lifts out from the casing 180 the auto injector 202 by means of the actuator end 202b of the auto injector 202. Where the auto injector 202 includes some kind of boot, cap or cover for sealing the injection end 202a and/or the auto injector's needle, the casing 180 can be configured to hold that boot, cap or cover. In this way, when the user lifts the auto injector 202 out of the casing, the boot, cap or cover is automatically removed from the auto injector.

With the adapter 212 still held in plate in the tray 182a of the lower portion 182, the user then offers the injection end 202a of the auto injector 202 to the outwardly facing adapter 212 such that the adapter 212 becomes fitted to the injection end 202a of the auto injector 202. Any appropriate fitting may be used, such as a simple friction fitting or the bayonet fitting discussed above. This arrangement is illustrated in FIG. 24.

As illustrated in FIG. 25, the user then uses the auto injector 202 to lift the adapter 212 out of the tray 182a of the lower portion 182 and offers the adapter 212 to the outwardly facing septum of the vial 222.

In a manner as described above, the adapter 212 is used to penetrate the septum of the vial 222 and attaches itself to the vial 222, for instance by means of the neck of the vial 222.

In this position, the auto injector 202 is operated to eject from it the pre-filled fluid, such as water, into the vial 222. In the illustrated embodiment, this is achieved by some internal power means, such as a spring. However, of course, manual operation could also be used.

In some embodiments, a mechanism may be provided such that the auto injector is responsive to fitment to a vial (via the adaptor) to trigger automatically the filling mechanism to eject the stored fluid into the vial.

In the next step, the user removes the auto injector 202 from the casing 180, taking with it the adapter 212 and vial 222. The auto injector 202 is then turned upside down and the actuator end 202b of the auto injector 202 is located in the receiving portion 192 of the tray 182a of the lower portion 182 as illustrated in FIG. 28. The receiving portion 192 is intended to grip the actuation end 202b of the auto injector 202 sufficiently securely to support and hold the auto injector 202 in an upright position with the adapter 212 vertically above the auto injector 202 and with the vial 222 vertically above the adapter 212.

With the assembled auto injector 202, adapter 212 and vial 222 in this orientation, the vial 222 can be left for some time as necessary to allow the fluid (water) which was injected into it to mix with the pre-filled substance to form the required medicament. It is to be noted that turning the assembly upside down to fit the actuation end 202b of the auto injector 202 in the tray can also assist in such mixing.

After an appropriate amount of time, the auto injector 202 is operated so as to withdraw the mixed medicament from the vial 222 into the auto injector 202. The orientation of the assembled auto injector 202, adapter 212 and vial 222 assists in this.

In the illustrated embodiment, like the embodiment described above, a sleeve 252 is provided for operating the filling mechanism. In particular, as illustrated in FIG. 27, the sleeve 252 is moved downwardly towards the lower portion 182 of the casing 180. With the auto injector 202 supported in the casing 180 in the upright position, this is an easy operation for the user.

In this embodiment, the sleeve 252 then exposes for the first time an actuation button 238a for actuating the deployment mechanism of the auto injector. Hence, the sleeve 252 acts as a lock-out for the deployment mechanism.

As illustrated in FIG. 28, the user now merely withdraws the assembled adapter 212 and 222 and stores this in the receiving portion 194 of the tray 182a of the lower portion 182 of the casing 180.

Having used the auto injector 202 as required, the auto injector 202 can then be relocated in its receiving portion 186 in the tray 182a of the lower portion 182 of the casing 180 as illustrated in FIG. 29, for instance for disposal.

1. An auto injector including:
a housing;
an outlet portion moveable relative to the housing;
a container within the housing for containing medicament;
an actuatable deployment mechanism configured to deploy the outlet portion by moving the outlet portion relative to the housing and to eject medicament contained in the container through the outlet portion; wherein:
the auto injector includes:
a filling mechanism configured to draw medicament through the outlet portion and into the container.

2. An auto injector according to claim 1 wherein the outlet portion includes a passageway.

3. An auto injector according to claim 2 wherein the outlet portion is configured to hold a needle such that the needle is in fluid communication with the passageway.

4. An auto injector according to claim 3 wherein the outlet portion includes the needle in fluid communication with the passageway.

5. An auto injector according to claim 2 wherein the container is in fluid communication with the passageway.

6. An auto injector according to claim 1 wherein the container includes:
walls defining an elongate bore extending from a container outlet; and
a piston sealing with the walls and slidable along the bore.

7. An auto injector according to claim 6 wherein the deployment mechanism is configured to eject medicament contained in the container by sliding the piston along the bore towards the container outlet.

8. An auto injector according to claim 6, wherein the filling mechanism is configured to draw medicament into the container by sliding the piston along the bore away from the container outlet.

9. An auto injector according to any preceding claim 1 wherein the filling mechanism includes a filling member moveable by a user from a first position to a second position to draw medicament through the outlet portion and into the container.

10. An auto injector according to claim 9 wherein the filling member is configured as a sleeve around the housing and is configured to slide along the housing from the first position to the second position.

11. An auto injector according to any preceding claim 1 wherein the deployment mechanism includes a user actuatable deployment actuator configured to actuate the deployment mechanism.

12. An auto injector according to claim 11 wherein the filling mechanism includes a lock out component for preventing operation of the user actuatable deployment actuator until the filling mechanism has drawn medicament into the container.

13. An auto injector according to claim 12 wherein the filling member is configured to cover the user actuatable actuator member in the first position and to expose the user actuatable deployment actuator in the second position.

14. An auto injector according to claim 1 wherein the auto injector is configured to store a volume of fluid and the filling mechanism is configured to expel the volume of fluid from the outlet portion before drawing medicament through the outlet portion and into the container.

15. An auto injector according to claim 14 wherein the container is configured to store the volume of the fluid.

16. An auto injector according to claim 15 further including water stored in the container.

17. (canceled)

18. An auto injector according to any one of claims 14 to 17 claim 14 further including:
an internal power source configured to power the filling mechanism to expel the volume of fluid.

19. An auto injector according to claim 18 wherein said power source is additionally configured to power the deployment mechanism.

20. An auto injector according to claim 18 wherein said power source is a spring.

21. An auto injector according to any one of claims 11 to 20 claim 14 wherein the filling mechanism includes a user actuatable filling actuator configured to actuate the filling mechanism to expel the volume of fluid.

22. An adaptor for use with an auto injector having a container having an elongate body with an injection end from which a needle extends to a needle tip, the adaptor including:
a housing arranged to mount on and seal with the container so as to define with the container a cavity within which the needle of the container can extend without the needle tip contacting the housing; and
a puncture member extending from the housing, arranged to puncture a septum of a vial sealing an interior of the vial and having at least one port arranged to provide fluid communication between the interior of the vial and the cavity.

23. An adaptor according to claim 22 wherein the housing includes a mounting portion for fitment to a housing of the auto injector to secure the adaptor to the auto injector.

24. An adaptor according to claim 23 wherein the mounting portion includes a collar for fitment to the housing of the auto injector and the housing of the adaptor extends within the collar to mount on and seal with the container of the auto injector.

25. An adaptor according to claim 23 wherein the mounting portion includes one of a bayonet fitting and a threaded fitting.

26. An auto injector set including:
an auto injector having a needle with which to inject medicament and
an adaptor configured to fit to the auto injector so as to cover the needle, the adaptor including a puncture member configured to puncture a vial, the puncture member providing fluid communication to the needle.

27. An auto injector set according to claim 26 wherein the adaptor includes a housing arranged to mount on and seal with the container so as to define with the container a cavity within which the needle of the container can extend without the needle tip contacting the housing; and wherein the puncture member extends from the housing, is arranged to puncture a septum of the vial sealing an interior of the vial and has at least one port arranged to provide fluid communication between the interior of the vial and the cavity.

28. An auto injector set according to claim 27 wherein the housing of the auto injector includes a receiving portion on which to fit the adaptor.

29. An auto injector according to claim 28 wherein the receiving portion includes one of a bayonet fitting and a threaded fitting.

30. An auto injector set according to claim 26 wherein the auto injector comprises:
a housing;
an outlet portion moveable relative to the housing:
a container within the housing for containing medicament;
an actuatable deployment mechanism configured to deploy the outlet portion by moving the outlet portion relative to the housing and to eject medicament contained in the container through the outlet portion; and
a filling mechanism configured to draw medicament through the outlet portion and into the container.

31. An auto injector pack including:
an auto injector set according to claim 26;
a vial containing a substance which in combination with said liquid forms the medicament; and
a casing configured to house the vial and also the auto injector and adaptor of the auto injector set.

32. An auto injector pack according to claim 31 wherein:
the casing includes a pre-sealed and sterilised section containing the vial and adaptor.

33. An auto injector pack according to claim 31 wherein:
the casing includes a lower portion having a shape to hold each of the auto injector, the vial and the adaptor individually, the lower portion having a shape to hold the adaptor facing outwardly so as to receive the needle of the auto injector and to hold the vial facing outwardly so as to receive the puncture member.

34. An auto injector pack according to claim 31 wherein:
the lower portion has a shape to hold the vial and the adaptor together as an assembled unit after the assembled unit has been detached from the auto injector.

35. An auto injector pack according to claim 31 wherein:
the lower portion has the form of a tray, the auto injector extends along an axis from the needle to an actuator end and the lower portion has a shape to hold the auto injector with the axis of the auto injector within the tray.

36. An auto injector pack according to claim 35 wherein the casing includes a presentation mechanism configured to lift the actuation end of the auto injector out of the lower portion upon opening of the casing.

37. An auto injector pack according to claim 35 wherein:
the lower portion is shaped to hold the actuation end of the auto injector with the auto injector orientated substantially perpendicular to the tray.

38. An auto injector pack according to claim 31 wherein the casing includes an upper portion forming a cover openable with respect to the lower portion.

39. A method of preparing the auto injector of the auto injector pack of claim 31, the method including:
removing the auto injector from the casing;
fitting the auto injector to the adaptor while the adaptor is in the casing;
removing the adaptor from the casing while fitted to the auto injector;
while the vial is in the casing, puncturing the vial with the puncture member of the adaptor and ejecting said fluid from the auto injector through the adaptor into the vial;
removing the vial from the casing while fitted to the adaptor and the auto injector;
re-orientating the auto injector and fitting the auto injector to the casing with the vial and adaptor above the auto injector;
withdrawing the medicament formed in the vial through the adaptor and into the auto injector; and
removing the adaptor and vial from the auto injector.

40. An auto injector according to claim 1
wherein the container includes walls defining an elongate bore extending from a container outlet, and a piston sealing with the walls and slidable along the bore; and
wherein the auto injector is configured to store a volume of fluid and the filling mechanism is configured to expel the volume of fluid from the outlet portion before drawing medicament through the outlet portion and into the container; and
wherein the filling mechanism is configured to expel the volume of fluid by sliding the piston along the bore towards the container outlet.
